(19) United States
(12) Patent Application Publication
(10) Pub. No.: US 2023/0151444 A1
(43) Pub. Date: May 18, 2023

(54) PCR BASED DIAGNOSTIC KIT, COMPOSITIONS AND METHODS FOR AMPLIFICATION AND DETECTION OF SARS-COV-2

(71) Applicant: FAST TRACK DIAGNOSTICS LUXEMBOURG S.A.R.L., Esch-sur-Alzette (LU)

(72) Inventors: Benedito EDUARDO CORREIA, Wilmington (DE); Alexandre Jean GILLES, Arlon (BE); Ludovic MENARD, Peppange (LU)

(21) Appl. No.: 17/995,543

(22) PCT Filed: Apr. 1, 2021
(86) PCT No.: PCT/EP2021/058556

§ 371 Date: Oct. 5, 2022

Related U.S. Application Data
(60) Provisional application No. 63/005,781, filed on Apr. 6, 2020.
(60) Provisional application No. 62/705,208, filed on Jun. 16, 2020.

(51) Int. Cl. C12Q 1/70; C12Q 1/686

(52) U.S. Cl. CPC C12Q 1/701; C12Q 1/686; C12Q 2600/16

(57) ABSTRACT

The present application is directed to a method for detecting presence or absence of severe acute respiratory syndrome coronavirus 2 (SARS-CoV-2) in a sample. The method first involves contacting the sample with a primary oligonucleotide primer set. The primary oligonucleotide primer set comprises: (i) a first oligonucleotide primer comprising a nucleotide sequence complementary to a first portion of the SARS-CoV-2 transmembrane domain 2 gene, and (ii) a second oligonucleotide primer comprising a nucleotide sequence complementary to an extension product formed from the first oligonucleotide primer of the primary oligonucleotide primer set. The method can further involve contacting the sample with a secondary oligonucleotide primer set, where the secondary primer set comprises (i) a first oligonucleotide primer comprising a nucleotide sequence complementary to a first portion of the SARS-CoV-2 N gene, and (ii) a second oligonucleotide primer comprising a nucleotide sequence complementary to an extension product formed from the first oligonucleotide primer of the secondary oligonucleotide primer set. The contacted sample is subjected to an amplification reaction under conditions suitable for producing transmembrane domain 2 gene and N gene amplification products, and the presence or absence of SARS-CoV-2 in the sample is detected. Isolated oligonucleotides, primer sets, and kits are also disclosed.

This application claims the benefit of U.S. Provisional Patent Application Serial Nos. 63/005,781, filed on Apr. 6, 2020, and 62/705,208, filed on Jun. 16, 2020, which are hereby incorporated by reference in their entirety.

### FIELD

The present application relates to a diagnostic kit, compositions and methods for amplification and detection of SARS-CoV-2.

### BACKGROUND

The severe acute respiratory coronavirus 2 (SARS-CoV-2), which emerged in December 2019 in Wuhan, China, has spread rapidly worldwide. The World Health Organization (WHO) called the disease caused by the virus, COVID-19. The outbreak was declared a Public Health Emergency of International Concern on Jan. 30, 2020, and a pandemic on Mar. 11, 2020 by WHO. As of May 18, 2020, the John Hopkins University Coronavirus Resource Center reported 4,730,323 confirmed cases worldwide and 315,482 deaths. This virus is reported to spread directly from person-to-person contact through respiratory droplets (such as coughing) or possibly through contaminated surfaces.

The first symptoms of the COVID-19 are not very specific. People may experience runny nose, headache, muscle pain and tiredness. Fever, cough and respiratory symptoms often occur two or three days later and can lead to severe pneumonia and death. The severity of clinical symptoms requires that approximately 20% of patients remain in hospital and 5% require admission to intensive care. The most serious forms are observed mainly in people who are vulnerable because of their age (over 70) or associated diseases. However, the infection can also be asymptomatic or paucisymptomatic (causing little or no clinical manifestations) in 30% to 60% of infected subjects. The duration of incubation is on average 5 days, with extremes of 2 to 12 days. More critically, it has been reported that a person showing no symptoms can transmit the virus to others, thus showing the importance of developing a sensitive and reliable test to detect SARS-CoV-2 to help save lives by limiting the spread of SARS-CoV-2.

SARS-CoV-2 belongs to the large family of Coronaviridae (genus Betacoronavirus). SARS-CoV-2 is genetically similar to SARS coronavirus and bat SARS-like coronaviruses. It is a positive-sense single-stranded RNA. Although bats are the likely reservoir hosts for SARS-CoV-2, there is still ongoing research investigating if pangolins (Manis javanica) are a possible intermediate host for this novel human virus (Lam et al., “Identifying SARS-CoV-2 Related Coronaviruses in Malayan Pangolins,” Nature doi.org/10.1038/s41586-020-2169-0 (2020)). SARS-CoV-2 is unique among known betacoronaviruses in its incorporation of a polybasic cleavage site, a characteristic known to increase pathogenicity and transmissibility in other viruses (Andersen et al., “The Proximal Origin of SARS-CoV-2,” Nature Medicine 26:450-452 (2020); Walls et al., “Structure, Function, and Antigenicity of the SARS-CoV-2 Spike Glycoprotein,” Cell 181(2):281-292 (2020); Coutard et al., “The Spike Glycoprotein of the New Coronavirus 2019-nCoV Contains a Furin-like Cleavage Site Absent in CoV of the Same Clade,” Antiviral Research 176:104742 (2020)).

Currently, there are two broad categories of SARS-CoV-2 diagnostic tests. The first category includes molecular assays, such as polymerase chain reaction (PCR), for detecting the virus itself, and the second category includes immunoassays for detecting the host's response to the virus (Patel et al., “Report from the American Society for Microbiology COVID-19 International Summit, 23 Mar. 2020: Value of Diagnostic Testing for SARS-CoV-2/COVID-19,” mBio 11(2): e00722-20 (2020).

As the development of an antibody response to infection takes time, antibody testing is not useful for detecting infection in asymptomatic patients or in patients in the early stage of acute illness. Thus, not only are molecular assays to detect the presence of the virus in a sample more sensitive, but they are also a better option for early detection of infection which is necessary to effectively prevent the spread of COVID-19.

Currently, most of the available developed SARS-CoV-2 PCR tests are developed to detect multiple regions of the SARS-CoV-2 genome, with each target amplicon giving an output signal to a different detection channel (e.g., all amplicons of a first target region of SARS-CoV-2 produce a signal in a first channel and all amplicons of a second target region of SARS-CoV-2 produce a signal in a second channel). A limitation of single target detection per channel is the potential lack of robustness as genetic polymorphism or potential mutations could compromise virus detection, and thus potentially lead to false negative results (Nagy et al., “Evaluation of TaqMan qPCR System Integrating Two Identically Labelled Hydrolysis Probes in Single Assay,” Scientific Reports 7:41392 (2017)). The failure to detect virus in infected patients is a major concern in a pandemic situation as it prevents efficient containment of the virus and can provoke secondary infection sites or second “waves” of infection.

The present application is directed at overcoming these and other deficiencies in the art.

### SUMMARY

A first aspect of the present application is directed to a method for detecting the presence or absence of severe acute respiratory syndrome coronavirus 2 (SARS-CoV-2) in a sample. This method involves contacting the sample with a primary oligonucleotide primer set, where the primary oligonucleotide primer set comprises (i) a first oligonucleotide primer comprising a nucleotide sequence complementary to a first portion of the SARS-CoV-2 transmembrane domain 2 gene of the open reading frame 1a (ORF1a), and (ii) a second oligonucleotide primer comprising a nucleotide sequence complementary to an extension product formed from the first oligonucleotide primer of the primary oligonucleotide primer set. The contacted sample is then subjected to an amplification reaction under conditions suitable for producing transmembrane domain 2 gene amplification products, and the presence or absence of SARS-CoV-2 in the sample is detected based on the production of those amplification products.

Another aspect of the present disclosure is directed to a method for detecting the presence or absence of SARS-CoV-2 in a sample that involves contacting the sample with the primary oligonucleotide primer set described above, and a secondary oligonucleotide primer set. The secondary oligonucleotide primer set comprises (i) a first oligonucleotide primer comprising a nucleotide sequence complementary to a first portion of the SARS-CoV-2 N gene, and (ii) a second oligonucleotide primer comprising a nucleotide sequence complementary to an extension product formed from the first oligonucleotide primer of the secondary oligonucleotide primer set. In accordance with this aspect, the amplification reaction is carried out under conditions suitable for producing transmembrane domain 2 and N gene amplification products, and the presence or absence of SARS-CoV-2 is detected based on the production of those amplification products.

Another aspect of the present application is directed to an isolated oligonucleotide suitable for detecting SARS-CoV-2. The isolated oligonucleotide comprises a nucleotide sequence selected from the group consisting of SEQ ID NO: 1, SEQ ID NO: 2, SEQ ID NO: 3, SEQ ID NO: 4, SEQ ID NO: 5, and SEQ ID NO: 6.

Another aspect of the present application is directed to an oligonucleotide primer set for detecting SARS-CoV-2 transmembrane domain 2 gene. The oligonucleotide primer set comprises a first oligonucleotide primer comprising the nucleotide sequence of SEQ ID NO: 1, and a second oligonucleotide primer comprising the nucleotide sequence of SEQ ID NO: 2.

Another aspect of the present application is directed to an oligonucleotide primer set for detecting SARS-CoV-2 N gene. The oligonucleotide primer set comprises a first oligonucleotide primer comprising the nucleotide sequence of SEQ ID NO: 4, and a second oligonucleotide primer comprising the nucleotide sequence of SEQ ID NO: 5.

The present application discloses a real-time reverse transcription polymerase chain reaction (RT-PCR) that provides a solution to the clinical need for a sensitive assay specific for the detection of SARS-CoV-2 in a biological sample. This assay features oligonucleotides which are suitable for determining whether SARS-CoV-2 is qualitatively present in a test sample (e.g., a nasopharyngeal sample) obtained from an individual suspected of having COVID-19. A dual-target assay with identically labelled probes is provided to circumvent the potential issue of false negative results due to genetic polymorphisms or potential mutations. Thus, the disclosed assay better guarantees inclusivity of the assay in the future.

### BRIEF DESCRIPTION OF THE DRAWINGS

FIG. 1 is a schematic illustrating the relative positions of amplicon targets on SARS-CoV-2. The two SARS-CoV-2 genomic regions targeted by the two different SARS-CoV-2 primer and probe sets are located (i) in the opening reading frame lab (ORF1ab) region (region coding for the Transmembrane domain 2 (TM2) gene of ORF1a) located at position 9928-10007 within the complete SARS-CoV-2 genome sequence, and (ii) in the N gene (coding for the Nucleo-capsid phosphoprotein) located at position 29257-29339 within the complete SARS-CoV-2 genome sequence. The expected size of amplicons are as follows: 80 base pairs for the target in ORF1ab region and 83 base pairs for the target in N gene.

FIGS. 2A-2C are tables showing the results (Ct values) of a study comparing sensitivity of the SARS-CoV-2 detection assay described in the present application (“Test Method”) to the Seegene SARS-CoV-2 detection assay (FIG. 2A), the Roche SARS-CoV-2 detection assay (FIG. 2B), and the Vircell SARS-CoV-2 detection assay (FIG. 2C).

### DETAILED DESCRIPTION

The present disclosure is directed to methods and reagents suitable for detecting severe acute respiratory coronavirus 2 (SARS-CoV-2), which emerged in December 2019 in Wuhan, China. SARS-CoV-2 comprises a single-stranded RNA genome that varies in size from 29.8 kb to 29.9 kb. The first sequence of the SARS-CoV-2 genome isolated from Wuhan was deposited in Genbank as accession no. NC_045512. The genomic structure of SARS-CoV-2 is characteristic of other known coronaviruses. In particular, more than two-thirds of the genome comprises the ORF1ab region (comprising ORF1a and ORF1b), which is located at the 5′ end of the genome and encodes ORF1ab polyproteins. The remaining one third of the genome, located 3′ to the ORF1ab region, consists of genes encoding structural proteins including surface (S), envelope (E), membrane (M), and nucleocapsid (N) proteins. Additionally, the SARS-CoV-2 contains six accessory proteins, encoded by ORF3a, ORF6, ORF7a, ORF7b, and ORF8 regions. The methods of detecting SARS-CoV-2 as disclosed herein are achieved by detecting at least a first unique region of the SARS-CoV-2 genomic RNA within ORF1a known as the transmembrane domain 2 gene. This gene region is detected alone or together with a second unique region SARS-CoV-2 genomic RNA located within the N gene.

Accordingly, a first aspect of the present application is directed to a method for detecting the presence or absence of SARS-CoV-2 in a sample. This method involves contacting the sample with a primary oligonucleotide primer set. The primary oligonucleotide primer set comprises: (i) a first oligonucleotide primer comprising a nucleotide sequence complementary to a first portion of the SARS-CoV-2 transmembrane domain 2 (TM2) gene of ORF1a, and (ii) a second oligonucleotide primer comprising a nucleotide sequence complementary to an extension product formed from the first oligonucleotide primer. The contacted sample is subjected to an amplification reaction under conditions suitable for producing TM2 gene amplification products, and the presence or absence of SARS-CoV-2 in the sample is detected based on the production of those TM2 gene amplification products.

As described above, the first characterized SARS-CoV-2 genomic RNA sequence is that which was deposited with Genbank and accorded the accession number NC_045512. While the primers and probes described herein were designed in reference to this genomic sequence, it is understood that the methods of detecting SARS-CoV-2 as described herein are not limited to the detection of only this isolate of the virus, but also encompass the detection of other isolates and natural variants of the SARS-CoV-2 virus. To date there are over 3500 genomic sequences of SARS-CoV-2 isolates found in GenBank, and the methods disclosed herein are suitable for detecting the presence of each of these genomic sequences in a sample.

A natural variant of SARS-CoV-2 has a sequence that is different from the genomic sequence of SARS-CoV-2 (Wuhan isolate) due to one or more naturally occurred mutations, including, but not limited to, point mutations, rearrangements, insertions, deletions, etc., to the genomic sequence that may or may not result in a phenotypic change. In some embodiments, variants of SARS-CoV-2 detected using the methods disclosed comprise at least 75% sequence similarity to the genome of the Wuhan isolate, at least 80% sequence similarity to the genome of the Wuhan isolate, at least 85% sequence similarity to the genome of the Wuhan isolate, at least 90% sequence similarity to the genome of the Wuhan isolate, at least 95% sequence similarity to the genome of the Wuhan isolate, or >95% sequence similarity to the genome of the Wuhan isolate.

As used herein, “sample” refers to any biological sample potentially containing the genomic RNA of the SARS-CoV-2. In some embodiments, the biological sample is a biological fluid or biological tissue. Biological fluid samples that can be subjected to the methods disclosed herein include, without limitation, a nasopharyngeal sample, an oropharyngeal sample, a saliva sample. Other suitable biological fluid samples include, urine, blood, plasma, serum, semen, stool, sputum, cerebrospinal fluid, tears, mucus, amniotic fluid, and the like. A biological tissue sample is a sample comprising a specific type or types of cell aggregate(s) (combined with those intercellular substances that form one of the structural materials of human, animal, plant, bacterial, fungal or viral structure). Examples of biological tissue samples that can be subjected to the methods disclosed herein include, without limitation, tissue biopsies or individual cell(s). In the case of a biological sample, the sample may be a crude sample or a processed sample obtained after various processing or preparation of the original sample.

As described above, the SARS-CoV-2 genome is a single-stranded RNA genome. Therefore, in some embodiments it is beneficial or otherwise necessary to extract or isolated RNA from the sample prior to or for analysis. RNA molecules can be isolated from cells and tissue and quantified using methods known in the art, e.g., guanidinium-acid-phenol extraction, density gradient centrifugation using cesium chloride or cesium trifluoroacetate, glass fiber filtration, and magnetic bead separation, with the particular extraction procedure chosen based on the sample. In some instances, with some techniques, it may also be possible to analyze the nucleic acid without extracting RNA from the sample.

In practicing the methods of the present application, the SARS-CoV-2 RNA or portions thereof are reverse-transcribed to synthesize complementary DNA (cDNA), which is then amplified and detected or directly detected. Reverse transcription of the SARS-CoV-2 RNA or portions thereof can be achieved using a reverse transcriptase enzyme (e.g., avian myeloblastosis virus reverse transcriptase or moloney murine leukemia virus reverse transcriptase), a mixture of deoxyribonucleotides, and the appropriate buffers and reaction conditions which are well known to those of skill in the art. In some embodiments, the reverse transcription reaction is primed using random hexamer primers or oligo(dT) primers. In some embodiments, the reverse transcription reaction is primed using gene specific primers. For example, in some embodiments, the reverse transcription reaction is primed using the first primer of the primary oligonucleotide primer set as described herein, i.e., a primer comprising a sequence that is complementary to a region of the TM2 gene within ORF1a. In some embodiments, the reverse transcription reaction is primed using the first primer of the secondary oligonucleotide primer set as described herein, i.e., a primer comprising a nucleotide sequence that is complementary to a region of the N gene. In some embodiments, the reverse transcription reaction is primed using the first primer of the primary oligonucleotide primer set and the first primer of the secondary oligonucleotide primer set as described herein. Thus, in some embodiments, the sample is a sample comprising the reverse transcription product of the SARS-CoV-2 genomic RNA.

Reverse transcription can be performed alone or in combination with an amplification step, e.g., reverse transcription polymerase chain reaction, which may be further modified to be quantitative, e.g., quantitative real time RT-PCR as described in U.S. Pat. No. 5,639,606 and Holland et al., Proc Natl Acad Sci USA 88(16):7276 (1991), which are hereby incorporated by reference in their entirety. Suitable amplification reaction processes are described in more detail infra.

The nucleic acid sequence of the ORF1ab region of SARS-CoV-2 is provided below as SEQ ID NO: 10 (Genbank Accession No. QHD43415.1; UniProt ID No. P0DTC1; Wu et al., “A New Coronavirus Associated with Human Respiratory Disease in China” Nature 579(7798):265-269(2020), which is hereby incorporated by reference in its entirety).

auggagagcc uugucccugg uuucaacgag aaaacacacg uccaacucag uuugccuguu 60
uuacagguuc gcgacgugcu cguacguggc uuuggagacu ccguggagga ggucuuauca 120
gaggcacguc aacaucuuaa agauggcacu uguggcuuag uagaaguuga aaaaggcguu 180
uugccucaac uugaacagcc cuauguguuc aucaaacguu cggaugcucg aacugcaccu 240
cauggucaug uuaugguuga gcugguagca gaacucgaag gcauucagua cggucguagu 300
ggugagacac uugguguccu ugucccucau gugggcgaaa uaccaguggc uuaccgcaag 360
guucuucuuc guaagaacgg uaauaaagga gcugguggcc auaguuacgg cgccgaucua 420
aagucauuug acuuaggcga cgagcuuggc acugauccuu augaagauuu ucaagaaaac 480
uggaacacua aacauagcag ugguguuacc cgugaacuca ugcgugagcu uaacggaggg 540
gcauacacuc gcuaugucga uaacaacuuc uguggcccug auggcuaccc ucuugagugc 600
auuaaagacc uucuagcacg ugcugguaaa gcuucaugca cuuuguccga acaacuggac 660
uuuauugaca cuaagagggg uguauacugc ugccgugaac augagcauga aauugcuugg 720
uacacggaac guucugaaaa gagcuaugaa uugcagacac cuuuugaaau uaaauuggca 780
aagaaauuug acaccuucaa uggggaaugu ccaaauuuug uauuucccuu aaauuccaua 840
aucaagacua uucaaccaag gguugaaaag aaaaagcuug auggcuuuau ggguagaauu 900
cgaucugucu auccaguugc gucaccaaau gaaugcaacc aaaugugccu uucaacucuc 960
augaagugug aucauugugg ugaaacuuca uggcagacgg gcgauuuugu uaaagccacu 1020
ugcgaauuuu guggcacuga gaauuugacu aaagaaggug ccacuacuug ugguuacuua 1080
ccccaaaaug cuguuguuaa aauuuauugu ccagcauguc acaauucaga aguaggaccu 1140
gagcauaguc uugccgaaua ccauaaugaa ucuggcuuga aaaccauucu ucguaagggu 1200
ggucgcacua uugccuuugg aggcugugug uucucuuaug uugguugcca uaacaagugu 1260
gccuauuggg uuccacgugc uagcgcuaac auagguugua accauacagg uguuguugga 1320
gaagguuccg aaggucuuaa ugacaaccuu cuugaaauac uccaaaaaga gaaagucaac 1380
aucaauauug uuggugacuu uaaacuuaau gaagagaucg ccauuauuuu ggcaucuuuu 1440
ucugcuucca caagugcuuu uguggaaacu gugaaagguu uggauuauaa agcauucaaa 1500
caaauuguug aauccugugg uaauuuuaaa guuacaaaag gaaaagcuaa aaaaggugcc 1560
uggaauauug gugaacagaa aucaauacug aguccucuuu augcauuugc aucagaggcu 1620
gcucguguug uacgaucaau uuucucccgc acucuugaaa cugcucaaaa uucugugcgu 1680
guuuuacaga aggccgcuau aacaauacua gauggaauuu cacaguauuc acugagacuc 1740
auugaugcua ugauguucac aucugauuug gcuacuaaca aucuaguugu aauggccuac 1800
auuacaggug guguuguuca guugacuucg caguggcuaa cuaacaucuu uggcacuguu 1860
uaugaaaaac ucaaacccgu ccuugauugg cuugaagaga aguuuaagga agguguagag 1920
uuucuuagag acgguuggga aauuguuaaa uuuaucucaa ccugugcuug ugaaauuguc 1980
gguggacaaa uugucaccug ugcaaaggaa auuaaggaga guguucagac auucuuuaag 2040
cuuguaaaua aauuuuuggc uuugugugcu gacucuauca uuauuggugg agcuaaacuu 2100
aaagccuuga auuuagguga aacauuuguc acgcacucaa agggauugua cagaaagugu 2160
guuaaaucca gagaagaaac uggccuacuc augccucuaa aagccccaaa agaaauuauc 2220
uucuuagagg gagaaacacu ucccacagaa guguuaacag aggaaguugu cuugaaaacu 2280
ggugauuuac aaccauuaga acaaccuacu agugaagcug uugaagcucc auugguuggu 2340
acaccaguuu guauuaacgg gcuuauguug cucgaaauca aagacacaga aaaguacugu 2400
gcccuugcac cuaauaugau gguaacaaac aauaccuuca cacucaaagg cggugcacca 2460
acaaagguua cuuuugguga ugacacugug auagaagugc aagguuacaa gagugugaau 2520
aucacuuuug aacuugauga aaggauugau aaaguacuua augagaagug cucugccuau 2580
acaguugaac ucgguacaga aguaaaugag uucgccugug uuguggcaga ugcugucaua 2640
aaaacuuugc aaccaguauc ugaauuacuu acaccacugg gcauugauuu agaugagugg 2700
aguauggcua cauacuacuu auuugaugag ucuggugagu uuaaauuggc uucacauaug 2760
uauuguucuu ucuacccucc agaugaggau gaagaagaag gugauuguga agaagaagag 2820
uuugagccau caacucaaua ugaguauggu acugaagaug auuaccaagg uaaaccuuug 2880
gaauuuggug ccacuucugc ugcucuucaa ccugaagaag agcaagaaga agauugguua 2940
gaugaugaua gucaacaaac uguuggucaa caagacggca gugaggacaa ucagacaacu 3000
acuauucaaa caauuguuga gguucaaccu caauuagaga uggaacuuac accaguuguu 3060
cagacuauug aagugaauag uuuuaguggu uauuuaaaac uuacugacaa uguauacauu 3120
aaaaaugcag acauugugga agaagcuaaa aagguaaaac caacaguggu uguuaaugca 3180
gccaauguuu accuuaaaca uggaggaggu guugcaggag ccuuaaauaa ggcuacuaac 3240
aaugccaugc aaguugaauc ugaugauuac auagcuacua auggaccacu uaaagugggu 3300
gguaguugug uuuuaagcgg acacaaucuu gcuaaacacu gucuucaugu ugucggccca 3360
aauguuaaca aaggugaaga cauucaacuu cuuaagagug cuuaugaaaa uuuuaaucag 3420
cacgaaguuc uacuugcacc auuauuauca gcugguauuu uuggugcuga cccuauacau 3480
ucuuuaagag uuuguguaga uacuguucgc acaaaugucu acuuagcugu cuuugauaaa 3540
aaucucuaug acaaacuugu uucaagcuuu uuggaaauga agagugaaaa gcaaguugaa 3600
caaaagaucg cugagauucc uaaagaggaa guuaagccau uuauaacuga aaguaaaccu 3660
ucaguugaac agagaaaaca agaugauaag aaaaucaaag cuuguguuga agaaguuaca 3720
acaacucugg aagaaacuaa guuccucaca gaaaacuugu uacuuuauau ugacauuaau 3780
ggcaaucuuc auccagauuc ugccacucuu guuagugaca uugacaucac uuucuuaaag 3840
aaagaugcuc cauauauagu gggugauguu guucaagagg guguuuuaac ugcugugguu 3900
auaccuacua aaaaggcugg uggcacuacu gaaaugcuag cgaaagcuuu gagaaaagug 3960
ccaacagaca auuauauaac cacuuacccg ggucaggguu uaaaugguua cacuguagag 4020
gaggcaaaga cagugcuuaa aaaguguaaa agugccuuuu acauucuacc aucuauuauc 4080
ucuaaugaga agcaagaaau ucuuggaacu guuucuugga auuugcgaga aaugcuugca 4140
caugcagaag aaacacgcaa auuaaugccu gucugugugg aaacuaaagc cauaguuuca 4200
acuauacagc guaaauauaa ggguauuaaa auacaagagg gugugguuga uuauggugcu 4260
agauuuuacu uuuacaccag uaaaacaacu guagcgucac uuaucaacac acuuaacgau 4320
cuaaaugaaa cucuuguuac aaugccacuu ggcuauguaa cacauggcuu aaauuuggaa 4380
gaagcugcuc gguauaugag aucucucaaa gugccagcua caguuucugu uucuucaccu 4440
gaugcuguua cagcguauaa ugguuaucuu acuucuucuu cuaaaacacc ugaagaacau 4500
uuuauugaaa ccaucucacu ugcugguucc uauaaagauu gguccuauuc uggacaaucu 4560
acacaacuag guauagaauu ucuuaagaga ggugauaaaa guguauauua cacuaguaau 4620
ccuaccacau uccaccuaga uggugaaguu aucaccuuug acaaucuuaa gacacuucuu 4680
ucuuugagag aagugaggac uauuaaggug uuuacaacag uagacaacau uaaccuccac 4740
acgcaaguug uggacauguc aaugacauau ggacaacagu uugguccaac uuauuuggau 4800
ggagcugaug uuacuaaaau aaaaccucau aauucacaug aagguaaaac auuuuauguu 4860
uuaccuaaug augacacucu acguguugag gcuuuugagu acuaccacac aacugauccu 4920
aguuuucugg guagguacau gucagcauua aaucacacua aaaaguggaa auacccacaa 4980
guuaaugguu uaacuucuau uaaaugggca gauaacaacu guuaucuugc cacugcauug 5040
uuaacacucc aacaaauaga guugaaguuu aauccaccug cucuacaaga ugcuuauuac 5100
agagcaaggg cuggugaagc ugcuaacuuu ugugcacuua ucuuagccua cuguaauaag 5160
acaguaggug aguuagguga uguuagagaa acaaugaguu acuuguuuca acaugccaau 5220
uuagauucuu gcaaaagagu cuugaacgug guguguaaaa cuuguggaca acagcagaca 5280
acccuuaagg guguagaagc uguuauguac augggcacac uuucuuauga acaauuuaag 5340
aaagguguuc agauaccuug uacguguggu aaacaagcua caaaauaucu aguacaacag 5400
gagucaccuu uuguuaugau gucagcacca ccugcucagu augaacuuaa gcaugguaca 5460
uuuacuugug cuagugagua cacugguaau uaccagugug gucacuauaa acauauaacu 5520
ucuaaagaaa cuuuguauug cauagacggu gcuuuacuua caaaguccuc agaauacaaa 5580
gguccuauua cggauguuuu cuacaaagaa aacaguuaca caacaaccau aaaaccaguu 5640
acuuauaaau uggauggugu uguuuguaca gaaauugacc cuaaguugga caauuauuau 5700
aagaaagaca auucuuauuu cacagagcaa ccaauugauc uuguaccaaa ccaaccauau 5760
ccaaacgcaa gcuucgauaa uuuuaaguuu guaugugaua auaucaaauu ugcugaugau 5820
uuaaaccagu uaacugguua uaagaaaccu gcuucaagag agcuuaaagu uacauuuuuc 5880
ccugacuuaa auggugaugu gguggcuauu gauuauaaac acuacacacc cucuuuuaag 5940
aaaggagcua aauuguuaca uaaaccuauu guuuggcaug uuaacaaugc aacuaauaaa 6000
gccacguaua aaccaaauac cugguguaua cguugucuuu ggagcacaaa accaguugaa 6060
acaucaaauu cguuugaugu acugaaguca gaggacgcgc agggaaugga uaaucuugcc 6120
ugcgaagauc uaaaaccagu cucugaagaa guaguggaaa auccuaccau acagaaagac 6180
guucuugagu guaaugugaa aacuaccgaa guuguaggag acauuauacu uaaaccagca 6240
aauaauaguu uaaaaauuac agaagagguu ggccacacag aucuaauggc ugcuuaugua 6300
gacaauucua gucuuacuau uaagaaaccu aaugaauuau cuagaguauu agguuugaaa 6360
acccuugcua cucaugguuu agcugcuguu aauagugucc cuugggauac uauagcuaau 6420
uaugcuaagc cuuuucuuaa caaaguuguu aguacaacua cuaacauagu uacacggugu 6480
uuaaaccgug uuuguacuaa uuauaugccu uauuucuuua cuuuauugcu acaauugugu 6540
acuuuuacua gaaguacaaa uucuagaauu aaagcaucua ugccgacuac uauagcaaag 6600
aauacuguua agagugucgg uaaauuuugu cuagaggcuu cauuuaauua uuugaaguca 6660
ccuaauuuuu cuaaacugau aaauauuaua auuugguuuu uacuauuaag uguuugccua 6720
gguucuuuaa ucuacucaac cgcugcuuua gguguuuuaa ugucuaauuu aggcaugccu 6780
ucuuacugua cugguuacag agaaggcuau uugaacucua cuaaugucac uauugcaacc 6840
uacuguacug guucuauacc uuguaguguu ugucuuagug guuuagauuc uuuagacacc 6900
uauccuucuu uagaaacuau acaaauuacc auuucaucuu uuaaauggga uuuaacugcu 6960
uuuggcuuag uugcagagug guuuuuggca uauauucuuu ucacuagguu uuucuaugua 7020
cuuggauugg cugcaaucau gcaauuguuu uucagcuauu uugcaguaca uuuuauuagu 7080
aauucuuggc uuaugugguu aauaauuaau cuuguacaaa uggccccgau uucagcuaug 7140
guuagaaugu acaucuucuu ugcaucauuu uauuauguau ggaaaaguua ugugcauguu 7200
guagacgguu guaauucauc aacuuguaug auguguuaca aacguaauag agcaacaaga 7260
gucgaaugua caacuauugu uaaugguguu agaagguccu uuuaugucua ugcuaaugga 7320
gguaaaggcu uuugcaaacu acacaauugg aauuguguua auugugauac auucugugcu 7380
gguaguacau uuauuaguga ugaaguugcg agagacuugu cacuacaguu uaaaagacca 7440
auaaauccua cugaccaguc uucuuacauc guugauagug uuacagugaa gaaugguucc 7500
auccaucuuu acuuugauaa agcuggucaa aagacuuaug aaagacauuc ucucucucau 7560
uuuguuaacu uagacaaccu gagagcuaau aacacuaaag guucauugcc uauuaauguu 7620
auaguuuuug augguaaauc aaaaugugaa gaaucaucug caaaaucagc gucuguuuac 7680
uacagucagc uuauguguca accuauacug uuacuagauc aggcauuagu gucugauguu 7740
ggugauagug cggaaguugc aguuaaaaug uuugaugcuu acguuaauac guuuucauca 7800
acuuuuaacg uaccaaugga aaaacucaaa acacuaguug caacugcaga agcugaacuu 7860
gcaaagaaug uguccuuaga caaugucuua ucuacuuuua uuucagcagc ucggcaaggg 7920
uuuguugauu cagauguaga aacuaaagau guuguugaau gucuuaaauu gucacaucaa 7980
ucugacauag aaguuacugg cgauaguugu aauaacuaua ugcucaccua uaacaaaguu 8040
gaaaacauga caccccguga ccuuggugcu uguauugacu guagugcgcg ucauauuaau 8100
gcgcagguag caaaaaguca caacauugcu uugauaugga acguuaaaga uuucauguca 8160
uugucugaac aacuacgaaa acaaauacgu agugcugcua aaaagaauaa cuuaccuuuu 8220
aaguugacau gugcaacuac uagacaaguu guuaauguug uaacaacaaa gauagcacuu 8280
aaggguggua aaauuguuaa uaauugguug aagcaguuaa uuaaaguuac acuuguguuc 8340
cuuuuuguug cugcuauuuu cuauuuaaua acaccuguuc augucauguc uaaacauacu 8400
gacuuuucaa gugaaaucau aggauacaag gcuauugaug guggugucac ucgugacaua 8460
gcaucuacag auacuuguuu ugcuaacaaa caugcugauu uugacacaug guuuagccag 8520
cgugguggua guuauacuaa ugacaaagcu ugcccauuga uugcugcagu cauaacaaga 8580
gaaguggguu uugucgugcc ugguuugccu ggcacgauau uacgcacaac uaauggugac 8640
uuuuugcauu ucuuaccuag aguuuuuagu gcaguuggua acaucuguua cacaccauca 8700
aaacuuauag aguacacuga cuuugcaaca ucagcuugug uuuuggcugc ugaauguaca 8760
auuuuuaaag augcuucugg uaagccagua ccauauuguu augauaccaa uguacuagaa 8820
gguucuguug cuuaugaaag uuuacgcccu gacacacguu augugcucau ggauggcucu 8880
auuauucaau uuccuaacac cuaccuugaa gguucuguua gagugguaac aacuuuugau 8940
ucugaguacu guaggcacgg cacuugugaa agaucagaag cugguguuug uguaucuacu 9000
agugguagau ggguacuuaa caaugauuau uacagaucuu uaccaggagu uuucuguggu 9060
guagaugcug uaaauuuacu uacuaauaug uuuacaccac uaauucaacc uauuggugcu 9120
uuggacauau cagcaucuau aguagcuggu gguauuguag cuaucguagu aacaugccuu 9180
gccuacuauu uuaugagguu uagaagagcu uuuggugaau acagucaugu aguugccuuu 9240
aauacuuuac uauuccuuau gucauucacu guacucuguu uaacaccagu uuacucauuc 9300
uuaccuggug uuuauucugu uauuuacuug uacuugacau uuuaucuuac uaaugauguu 9360
ucuuuuuuag cacauauuca guggaugguu auguucacac cuuuaguacc uuucuggaua 9420
acaauugcuu auaucauuug uauuuccaca aagcauuucu auugguucuu uaguaauuac 9480
cuaaagagac guguagucuu uaaugguguu uccuuuagua cuuuugaaga agcugcgcug 9540
ugcaccuuuu uguuaaauaa agaaauguau cuaaaguugc guagugaugu gcuauuaccu 9600
cuuacgcaau auaauagaua cuuagcucuu uauaauaagu acaaguauuu uaguggagca 9660
auggauacaa cuagcuacag agaagcugcu uguugucauc ucgcaaaggc ucucaaugac 9720
uucaguaacu cagguucuga uguucuuuac caaccaccac aaaccucuau caccucagcu 9780
guuuugcaga gugguuuuag aaaaauggca uucccaucug guaaaguuga ggguuguaug 9840
guacaaguaa cuugugguac aacuacacuu aacggucuuu ggcuugauga cguaguuuac 9900
uguccaagac augugaucug caccucugaa gacaugcuua acccuaauua ugaagauuua 9960
cucauucgua agucuaauca uaauuucuug guacaggcug guaauguuca acucaggguu 10020
auuggacauu cuaugcaaaa uuguguacuu aagcuuaagg uugauacagc caauccuaag 10080
acaccuaagu auaaguuugu ucgcauucaa ccaggacaga cuuuuucagu guuagcuugu 10140
uacaaugguu caccaucugg uguuuaccaa ugugcuauga ggcccaauuu cacuauuaag 10200
gguucauucc uuaaugguuc augugguagu guugguuuua acauagauua ugacuguguc 10260
ucuuuuuguu acaugcacca uauggaauua ccaacuggag uucaugcugg cacagacuua 10320
gaagguaacu uuuauggacc uuuuguugac aggcaaacag cacaagcagc ugguacggac 10380
acaacuauua caguuaaugu uuuagcuugg uuguacgcug cuguuauaaa uggagacagg 10440
ugguuucuca aucgauuuac cacaacucuu aaugacuuua accuuguggc uaugaaguac 10500
aauuaugaac cucuaacaca agaccauguu gacauacuag gaccucuuuc ugcucaaacu 10560
ggaauugccg uuuuagauau gugugcuuca uuaaaagaau uacugcaaaa ugguaugaau 10620
ggacguacca uauuggguag ugcuuuauua gaagaugaau uuacaccuuu ugauguuguu 10680
agacaaugcu cagguguuac uuuccaaagu gcagugaaaa gaacaaucaa ggguacacac 10740
cacugguugu uacucacaau uuugacuuca cuuuuaguuu uaguccagag uacucaaugg 10800
ucuuuguucu uuuuuuugua ugaaaaugcc uuuuuaccuu uugcuauggg uauuauugcu 10860
augucugcuu uugcaaugau guuugucaaa cauaagcaug cauuucucug uuuguuuuug 10920
uuaccuucuc uugccacugu agcuuauuuu aauauggucu auaugccugc uaguugggug 10980
augcguauua ugacaugguu ggauaugguu gauacuaguu ugucugguuu uaagcuaaaa 11040
gacuguguua uguaugcauc agcuguagug uuacuaaucc uuaugacagc aagaacugug 11100
uaugaugaug gugcuaggag aguguggaca cuuaugaaug ucuugacacu cguuuauaaa 11160
guuuauuaug guaaugcuuu agaucaagcc auuuccaugu gggcucuuau aaucucuguu 11220
acuucuaacu acucaggugu aguuacaacu gucauguuuu uggccagagg uauuguuuuu 11280
auguguguug aguauugccc uauuuucuuc auaacuggua auacacuuca guguauaaug 11340
cuaguuuauu guuucuuagg cuauuuuugu acuuguuacu uuggccucuu uuguuuacuc 11400
aaccgcuacu uuagacugac ucuugguguu uaugauuacu uaguuucuac acaggaguuu 11460
agauauauga auucacaggg acuacuccca cccaagaaua gcauagaugc cuucaaacuc 11520
aacauuaaau uguugggugu ugguggcaaa ccuuguauca aaguagccac uguacagucu 11580
aaaaugucag auguaaagug cacaucagua gucuuacucu caguuuugca acaacucaga 11640
guagaaucau caucuaaauu gugggcucaa uguguccagu uacacaauga cauucucuua 11700
gcuaaagaua cuacugaagc cuuugaaaaa augguuucac uacuuucugu uuugcuuucc 11760
augcagggug cuguagacau aaacaagcuu ugugaagaaa ugcuggacaa cagggcaacc 11820
uuacaagcua uagccucaga guuuaguucc cuuccaucau augcagcuuu ugcuacugcu 11880
caagaagcuu augagcaggc uguugcuaau ggugauucug aaguuguucu uaaaaaguug 11940
aagaagucuu ugaauguggc uaaaucugaa uuugaccgug augcagccau gcaacguaag 12000
uuggaaaaga uggcugauca agcuaugacc caaauguaua aacaggcuag aucugaggac 12060
aagagggcaa aaguuacuag ugcuaugcag acaaugcuuu ucacuaugcu uagaaaguug 12120
gauaaugaug cacucaacaa cauuaucaac aaugcaagag augguugugu ucccuugaac 12180
auaauaccuc uuacaacagc agccaaacua augguuguca uaccagacua uaacacauau 12240
aaaaauacgu gugaugguac aacauuuacu uaugcaucag cauuguggga aauccaacag 12300
guuguagaug cagauaguaa aauuguucaa cuuagugaaa uuaguaugga caauucaccu 12360
aauuuagcau ggccucuuau uguaacagcu uuaagggcca auucugcugu caaauuacag 12420
aauaaugagc uuaguccugu ugcacuacga cagaugucuu gugcugccgg uacuacacaa 12480
acugcuugca cugaugacaa ugcguuagcu uacuacaaca caacaaaggg agguagguuu 12540
guacuugcac uguuauccga uuuacaggau uugaaauggg cuagauuccc uaagagugau 12600
ggaacuggua cuaucuauac agaacuggaa ccaccuugua gguuuguuac agacacaccu 12660
aaagguccua aagugaagua uuuauacuuu auuaaaggau uaaacaaccu aaauagaggu 12720
augguacuug guaguuuagc ugccacagua cgucuacaag cugguaaugc aacagaagug 12780
ccugccaauu caacuguauu aucuuucugu gcuuuugcug uagaugcugc uaaagcuuac 12840
aaagauuauc uagcuagugg gggacaacca aucacuaauu guguuaagau guuguguaca 12900
cacacuggua cuggucaggc aauaacaguu acaccggaag ccaauaugga ucaagaaucc 12960
uuugguggug caucguguug ucuguacugc cguugccaca uagaucaucc aaauccuaaa 13020
ggauuuugug acuuaaaagg uaaguaugua caaauaccua caacuugugc uaaugacccu 13080
guggguuuua cacuuaaaaa cacagucugu accgucugcg guauguggaa agguuauggc 13140
uguaguugug aucaacuccg cgaacccaug cuucagucag cugaugcaca aucguuuuua 13200
aaccggguuu gcgguguaag ugcagcccgu cuuacaccgu gcggcacagg cacuaguacu 13260
gaugucguau acagggcuuu ugacaucuac aaugauaaag uagcugguuu ugcuaaauuc 13320
cuaaaaacua auuguugucg cuuccaagaa aaggacgaag augacaauuu aauugauucu 13380
uacuuuguag uuaagagaca cacuuucucu aacuaccaac augaagaaac aauuuauaau 13440
uuacuuaagg auuguccagc uguugcuaaa caugacuucu uuaaguuuag aauagacggu 13500
gacaugguac cacauauauc acgucaacgu cuuacuaaau acacaauggc agaccucguc 13560
uaugcuuuaa ggcauuuuga ugaagguaau ugugacacau uaaaagaaau acuugucaca 13620
uacaauuguu gugaugauga uuauuucaau aaaaaggacu gguaugauuu uguagaaaac 13680
ccagauauau uacgcguaua cgccaacuua ggugaacgug uacgccaagc uuuguuaaaa 13740
acaguacaau ucugugaugc caugcgaaau gcugguauug uugguguacu gacauuagau 13800
aaucaagauc ucaaugguaa cugguaugau uucggugauu ucauacaaac cacgccaggu 13860
aguggaguuc cuguuguaga uucuuauuau ucauuguuaa ugccuauauu aaccuugacc 13920
agggcuuuaa cugcagaguc acauguugac acugacuuaa caaagccuua cauuaagugg 13980
gauuuguuaa aauaugacuu cacggaagag agguuaaaac ucuuugaccg uuauuuuaaa 14040
uauugggauc agacauacca cccaaauugu guuaacuguu uggaugacag augcauucug 14100
cauugugcaa acuuuaaugu uuuauucucu acaguguucc caccuacaag uuuuggacca 14160
cuagugagaa aaauauuugu ugaugguguu ccauuuguag uuucaacugg auaccacuuc 14220
agagagcuag guguuguaca uaaucaggau guaaacuuac auagcucuag acuuaguuuu 14280
aaggaauuac uuguguaugc ugcugacccu gcuaugcacg cugcuucugg uaaucuauua 14340
cuagauaaac gcacuacgug cuuuucagua gcugcacuua cuaacaaugu ugcuuuucaa 14400
acugucaaac ccgguaauuu uaacaaagac uucuaugacu uugcuguguc uaaggguuuc 14460
uuuaaggaag gaaguucugu ugaauuaaaa cacuucuucu uugcucagga ugguaaugcu 14520
gcuaucagcg auuaugacua cuaucguuau aaucuaccaa caauguguga uaucagacaa 14580
cuacuauuug uaguugaagu uguugauaag uacuuugauu guuacgaugg uggcuguauu 14640
aaugcuaacc aagucaucgu caacaaccua gacaaaucag cugguuuucc auuuaauaaa 14700
ugggguaagg cuagacuuua uuaugauuca augaguuaug aggaucaaga ugcacuuuuc 14760
gcauauacaa aacguaaugu caucccuacu auaacucaaa ugaaucuuaa guaugccauu 14820
agugcaaaga auagagcucg caccguagcu ggugucucua ucuguaguac uaugaccaau 14880
agacaguuuc aucaaaaauu auugaaauca auagccgcca cuagaggagc uacuguagua 14940
auuggaacaa gcaaauucua uggugguugg cacaacaugu uaaaaacugu uuauagugau 15000
guagaaaacc cucaccuuau ggguugggau uauccuaaau gugauagagc caugccuaac 15060
augcuuagaa uuauggccuc acuuguucuu gcucgcaaac auacaacgug uuguagcuug 15120
ucacaccguu ucuauagauu agcuaaugag ugugcucaag uauugaguga aauggucaug 15180
uguggcgguu cacuauaugu uaaaccaggu ggaaccucau caggagaugc cacaacugcu 15240
uaugcuaaua guguuuuuaa cauuugucaa gcugucacgg ccaauguuaa ugcacuuuua 15300
ucuacugaug guaacaaaau ugccgauaag uauguccgca auuuacaaca cagacuuuau 15360
gagugucucu auagaaauag agauguugac acagacuuug ugaaugaguu uuacgcauau 15420
uugcguaaac auuucucaau gaugauacuc ucugacgaug cuguugugug uuucaauagc 15480
acuuaugcau cucaaggucu aguggcuagc auaaagaacu uuaagucagu ucuuuauuau 15540
caaaacaaug uuuuuauguc ugaagcaaaa uguuggacug agacugaccu uacuaaagga 15600
ccucaugaau uuugcucuca acauacaaug cuaguuaaac agggugauga uuauguguac 15660
cuuccuuacc cagauccauc aagaauccua ggggccggcu guuuuguaga ugauaucgua 15720
aaaacagaug guacacuuau gauugaacgg uucgugucuu uagcuauaga ugcuuaccca 15780
cuuacuaaac auccuaauca ggaguaugcu gaugucuuuc auuuguacuu acaauacaua 15840
agaaagcuac augaugaguu aacaggacac auguuagaca uguauucugu uaugcuuacu 15900
aaugauaaca cuucaaggua uugggaaccu gaguuuuaug aggcuaugua cacaccgcau 15960
acagucuuac aggcuguugg ggcuuguguu cuuugcaauu cacagacuuc auuaagaugu 16020
ggugcuugca uacguagacc auucuuaugu uguaaaugcu guuacgacca ugucauauca 16080
acaucacaua aauuagucuu gucuguuaau ccguauguuu gcaaugcucc agguugugau 16140
gucacagaug ugacucaacu uuacuuagga gguaugagcu auuauuguaa aucacauaaa 16200
ccacccauua guuuuccauu gugugcuaau ggacaaguuu uugguuuaua uaaaaauaca 16260
uguguuggua gcgauaaugu uacugacuuu aaugcaauug caacauguga cuggacaaau 16320
gcuggugauu acauuuuagc uaacaccugu acugaaagac ucaagcuuuu ugcagcagaa 16380
acgcucaaag cuacugagga gacauuuaaa cugucuuaug guauugcuac uguacgugaa 16440
gugcugucug acagagaauu acaucuuuca ugggaaguug guaaaccuag accaccacuu 16500
aaccgaaauu augucuuuac ugguuaucgu guaacuaaaa acaguaaagu acaaauagga 16560
gaguacaccu uugaaaaagg ugacuauggu gaugcuguug uuuaccgagg uacaacaacu 16620
uacaaauuaa auguugguga uuauuuugug cugacaucac auacaguaau gccauuaagu 16680
gcaccuacac uagugccaca agagcacuau guuagaauua cuggcuuaua cccaacacuc 16740
aauaucucag augaguuuuc uagcaauguu gcaaauuauc aaaagguugg uaugcaaaag 16800
uauucuacac uccagggacc accugguacu gguaagaguc auuuugcuau uggccuagcu 16860
cucuacuacc cuucugcucg cauaguguau acagcuugcu cucaugccgc uguugaugca 16920
cuaugugaga aggcauuaaa auauuugccu auagauaaau guaguagaau uauaccugca 16980
cgugcucgug uagaguguuu ugauaaauuc aaagugaauu caacauuaga acaguauguc 17040
uuuuguacug uaaaugcauu gccugagacg acagcagaua uaguugucuu ugaugaaauu 17100
ucaauggcca caaauuauga uuugaguguu gucaaugcca gauuacgugc uaagcacuau 17160
guguacauug gcgacccugc ucaauuaccu gcaccacgca cauugcuaac uaagggcaca 17220
cuagaaccag aauauuucaa uucagugugu agacuuauga aaacuauagg uccagacaug 17280
uuccucggaa cuugucggcg uuguccugcu gaaauuguug acacugugag ugcuuugguu 17340
uaugauaaua agcuuaaagc acauaaagac aaaucagcuc aaugcuuuaa aauguuuuau 17400
aaggguguua ucacgcauga uguuucaucu gcaauuaaca ggccacaaau aggcguggua 17460
agagaauucc uuacacguaa cccugcuugg agaaaagcug ucuuuauuuc accuuauaau 17520
ucacagaaug cuguagccuc aaagauuuug ggacuaccaa cucaaacugu ugauucauca 17580
cagggcucag aauaugacua ugucauauuc acucaaacca cugaaacagc ucacucuugu 17640
aauguaaaca gauuuaaugu ugcuauuacc agagcaaaag uaggcauacu uugcauaaug 17700
ucugauagag accuuuauga caaguugcaa uuuacaaguc uugaaauucc acguaggaau 17760
guggcaacuu uacaagcuga aaauguaaca ggacucuuua aagauuguag uaagguaauc 17820
acuggguuac auccuacaca ggcaccuaca caccucagug uugacacuaa auucaaaacu 17880
gaagguuuau guguugacau accuggcaua ccuaaggaca ugaccuauag aagacucauc 17940
ucuaugaugg guuuuaaaau gaauuaucaa guuaaugguu acccuaacau guuuaucacc 18000
cgcgaagaag cuauaagaca uguacgugca uggauuggcu ucgaugucga ggggugucau 18060
gcuacuagag aagcuguugg uaccaauuua ccuuuacagc uagguuuuuc uacagguguu 18120
aaccuaguug cuguaccuac agguuauguu gauacaccua auaauacaga uuuuuccaga 18180
guuagugcua aaccaccgcc uggagaucaa uuuaaacacc ucauaccacu uauguacaaa 18240
ggacuuccuu ggaauguagu gcguauaaag auuguacaaa uguuaaguga cacacuuaaa 18300
aaucucucug acagagucgu auuugucuua ugggcacaug gcuuugaguu gacaucuaug 18360
aaguauuuug ugaaaauagg accugagcgc accuguuguc uaugugauag acgugccaca 18420
ugcuuuucca cugcuucaga cacuuaugcc uguuggcauc auucuauugg auuugauuac 18480
gucuauaauc cguuuaugau ugauguucaa caaugggguu uuacagguaa ccuacaaagc 18540
aaccaugauc uguauuguca aguccauggu aaugcacaug uagcuaguug ugaugcaauc 18600
augacuaggu gucuagcugu ccacgagugc uuuguuaagc guguugacug gacuauugaa 18660
uauccuauaa uuggugauga acugaagauu aaugcggcuu guagaaaggu ucaacacaug 18720
guuguuaaag cugcauuauu agcagacaaa uucccaguuc uucacgacau ugguaacccu 18780
aaagcuauua aguguguacc ucaagcugau guagaaugga aguucuauga ugcacagccu 18840
uguagugaca aagcuuauaa aauagaagaa uuauucuauu cuuaugccac acauucugac 18900
aaauucacag augguguaug ccuauuuugg aauugcaaug ucgauagaua uccugcuaau 18960
uccauuguuu guagauuuga cacuagagug cuaucuaacc uuaacuugcc ugguugugau 19020
gguggcaguu uguauguaaa uaaacaugca uuccacacac cagcuuuuga uaaaagugcu 19080
uuuguuaauu uaaaacaauu accauuuuuc uauuacucug acaguccaug ugagucucau 19140
ggaaaacaag uagugucaga uauagauuau guaccacuaa agucugcuac guguauaaca 19200
cguugcaauu uagguggugc ugucuguaga caucaugcua augaguacag auuguaucuc 19260
gaugcuuaua acaugaugau cucagcuggc uuuagcuugu ggguuuacaa acaauuugau 19320
acuuauaacc ucuggaacac uuuuacaaga cuucagaguu uagaaaaugu ggcuuuuaau 19380
guuguaaaua agggacacuu ugauggacaa cagggugaag uaccaguuuc uaucauuaau 19440
aacacuguuu acacaaaagu ugaugguguu gauguagaau uguuugaaaa uaaaacaaca 19500
uuaccuguua auguagcauu ugagcuuugg gcuaagcgca acauuaaacc aguaccagag 19560
gugaaaauac ucaauaauuu ggguguggac auugcugcua auacugugau cugggacuac 19620
aaaagagaug cuccagcaca uauaucuacu auugguguuu guucuaugac ugacauagcc 19680
aagaaaccaa cugaaacgau uugugcacca cucacugucu uuuuugaugg uagaguugau 19740
ggucaaguag acuuauuuag aaaugcccgu aaugguguuc uuauuacaga agguaguguu 19800
aaagguuuac aaccaucugu aggucccaaa caagcuaguc uuaauggagu cacauuaauu 19860
ggagaagccg uaaaaacaca guucaauuau uauaagaaag uugauggugu uguccaacaa 19920
uuaccugaaa cuuacuuuac ucagaguaga aauuuacaag aauuuaaacc caggagucaa 19980
auggaaauug auuucuuaga auuagcuaug gaugaauuca uugaacggua uaaauuagaa 20040
ggcuaugccu ucgaacauau cguuuaugga gauuuuaguc auagucaguu aggugguuua 20100
caucuacuga uuggacuagc uaaacguuuu aaggaaucac cuuuugaauu agaagauuuu 20160
auuccuaugg acaguacagu uaaaaacuau uucauaacag augcgcaaac agguucaucu 20220
aagugugugu guucuguuau ugauuuauua cuugaugauu uuguugaaau aauaaaaucc 20280
caagauuuau cuguaguuuc uaagguuguc aaagugacua uugacuauac agaaauuuca 20340
uuuaugcuuu gguguaaaga uggccaugua gaaacauuuu acccaaaauu acaaucuagu 20400
caagcguggc aaccgggugu ugcuaugccu aaucuuuaca aaaugcaaag aaugcuauua 20460
gaaaagugug accuucaaaa uuauggugau agugcaacau uaccuaaagg cauaaugaug 20520
aaugucgcaa aauauacuca acugugucaa uauuuaaaca cauuaacauu agcuguaccc 20580
uauaauauga gaguuauaca uuuuggugcu gguucugaua aaggaguugc accagguaca 20640
gcuguuuuaa gacagugguu gccuacgggu acgcugcuug ucgauucaga ucuuaaugac 20700
uuugucucug augcagauuc aacuuugauu ggugauugug caacuguaca uacagcuaau 20760
aaaugggauc ucauuauuag ugauauguac gacccuaaga cuaaaaaugu uacaaaagaa 20820
aaugacucua aagaggguuu uuucacuuac auuugugggu uuauacaaca aaagcuagcu 20880
cuuggagguu ccguggcuau aaagauaaca gaacauucuu ggaaugcuga ucuuuauaag 20940
cucaugggac acuucgcaug guggacagcc uuuguuacua augugaaugc gucaucaucu 21000
gaagcauuuu uaauuggaug uaauuaucuu ggcaaaccac gcgaacaaau agaugguuau 21060
gucaugcaug caaauuacau auuuuggagg aauacaaauc caauucaguu gucuuccuau 21120
ucuuuauuug acaugaguaa auuuccccuu aaauuaaggg guacugcugu uaugucuuua 21180
aaagaagguc aaaucaauga uaugauuuua ucucuucuua guaaagguag acuuauaauu 21240
agagaaaaca acagaguugu uauuucuagu gauguucuug uuaacaacua a 21291

Two large replicase polyproteins, pp1a and pp1ab, which are encoded by ORF1ab, are proteolytically cleaved into 16 putative non-structural proteins (nsps) (Chan et al., “Genomic Characterization of the 2019 Novel Human-Pathogenic Coronavirus Isolated from a Patient with Atypical Pneumonia After Visiting Wuhan,” Emer. Microbes Infect. 9(1):221-236 (2020), which is hereby incorporated by reference in its entirety). These putative nsps include two viral cysteine proteases, namely, nsp3 (papain-like protease) and nsp5 (chymotrypsin-like, 3C-like, or main protease), along with nsp12 (RNA-dependent RNA polymerase [RARp]), nsp13 (helicase), and other nsps which are likely involved in the transcription and replication of the virus (Chan et al., “Genomic Characterization of the 2019 Novel Human-Pathogenic Coronavirus Isolated from a Patient with Atypical Pneumonia After Visiting Wuhan,” Emer. Microbes Infect. 9(1):221-236 (2020), which is hereby incorporated by reference in its entirety). The nsp4 encoding region, containing the transmembrane 2 domain (TM2) gene (see Snij der et al., “Unique and Conserved Features of Genome and Proteome of SARS-coronavirus, an Early Split-Off from the Coronavirus Group 2 Lineage,” J. Mol. Biol. 331(5):99-1004 (2003), which is hereby incorporated by reference in its entirety), is the region of SARS-CoV-2 detected using the methods described herein. The TM2 gene has the nucleotide sequence of SEQ ID NO: 12, which shows enough variabilities (relative to other viral gene sequences) to be specific to SARS-CoV-2. Thus, detection of the TM2 gene is selective for the detection of SARS-COV-2.

ggauacaacuagcuacagagaagcugcuuguugucaucucgcaaaggcucucaaugacuucaguaacucagguucugaugu (SEQ ID NO: 12)

In some embodiments, the method described herein involves the detection of the TM2 gene of SARS-CoV-2 alone. In some embodiments, the methods described herein involve the detection of the TM2 gene of SARS-CoV-2 in conjunction with the detection of at least one other region of SARS-CoV-2. In some embodiments, the second region of the SARS-CoV-2 RNA detected is a region within the N gene. Thus, one aspect of the present disclosure is directed to a method of detecting the presence or absence of SARS-CoV-2 in a sample that involves contacting the sample with the primary oligonucleotide primer set complementary to the TM2 gene of ORF1a together with at least a secondary oligonucleotide primer set. The secondary oligonucleotide primer set comprises (i) a first oligonucleotide primer comprising a nucleotide sequence complementary to a first portion of the SARS-CoV-2 N gene, and (ii) a second oligonucleotide primer comprising a nucleotide sequence complementary to an extension product formed from the first oligonucleotide primer of the secondary oligonucleotide primer set.

The nucleotide sequence of the N gene is provided below as SEQ ID NO:11 (Genbank Accession No. NC_045512 (28274 . . . 29533); Gene ID No. 43740575):

| | |
|---|---|
| 1 | augucugaua auggacccca aaaucagcga |
| | aaugcacccc gcauuacguu ugguggaccc |
| 61 | ucagauucaa cuggcaguaa ccagaaugga |
| | gaacgcagug gggcgcgauc aaaacaacgu |
| 121 | cggccccaag guuuacccaa uaauacugcg |
| | ucuugguuca ccgcucucac ucaacauggc |
| 181 | aaggaagacc uuaaauuccc ucgaggacaa |
| | ggcguuccaa uuaacaccaa uagcagucca |
| 241 | gaugaccaaa uuggcuacua ccgaagagcu |
| | accagacgaa uucguggugg ugacgguaaa |
| 301 | augaaagauc ucaguccaag augguauuuc |
| | uacuaccuag gaacugggcc agaagcugga |
| 361 | cuucccuaug gugcuaacaa agacggcauc |
| | auauggguug caacugaggg agccuugaau |
| 421 | acaccaaaag aucacauugg cacccgcaau |
| | ccugcuaaca augcugcaau cgugcuacaa |
| 481 | cuuccucaag gaacaacauu gccaaaaggc |
| | uucuacgcag aagggagcag aggcggcagu |
| 541 | caagccucuu cucguuccuc aucacguagu |
| | cgcaacaguu caagaaauuc aacuccaggc |
| 601 | agcaguaggg gaacuucucc ugcuagaaug |
| | gcuggcaaug gcggugaugc ugcucuugcu |
| 661 | uugcugcugc uugacagauu gaaccagcuu |
| | gagagcaaaa ugucugguaa aggccaacaa |
| 721 | caacaaggcc aaacugucac uaagaaaucu |
| | gcugcugagg cuucuaagaa gccucggcaa |
| 781 | aaacguacug ccacuaaagc auacaaugua |
| | acacaagcuu ucggcagacg ugguccagaa |
| 841 | caaacccaag gaaauuuugg ggaccaggaa |
| | cuaaucagac aaggaacuga uuacaaacau |
| 901 | uggccgcaaa uugcacaauu ugcccccagc |
| | gcuucagcgu ucuucggaau gucgcgcauu |
| 961 | ggcauggaag ucacaccuuc gggaacgugg |
| | uugaccuaca caggugccau caaauuggau |
| 1021 | gacaaagauc caaauuucaa agaucaaguc |
| | auuuugcuga auaagcauau ugacgcauac |
| 1081 | aaaacauucc caccaacaga gccuaaaaag |
| | gacaaaaaga agaaggcuga ugaaacucaa |
| 1141 | gccuuaccgc agagacagaa gaaacagcaa |
| | acugugacuc uucuuccugc ugcagauuug |
| 1201 | gaugauuucu ccaaacaauu gcaacaaucc |
| | augagcagug cugacucaac ucaggccuaa |

As used herein, an “oligonucleotide primer” refers to a nucleic acid molecule that hybridizes in a sequence specific manner to a complementary nucleic acid molecule (i.e., a target nucleic acid molecule) and is capable of initiating template-directed synthesis using methods such as polymerase chain reaction (PCR) under appropriate conditions (e.g., in the presence of four nucleotide triphosphates and a polymerase enzyme, such as DNA polymerase, reverse-transcriptase, etc., in an appropriate buffer solution containing any necessary reagents and at suitable temperature(s)). Such template directed synthesis is called primer extension and results in the generation of a primer extension product.

The oligonucleotide primers of the present disclosure can be in the form of ribonucleotides, deoxynucleotides, modified ribonucleotides, modified deoxyribonucleotides, modified phosphate-sugar-backbone oligonucleotides, nucleotide analogs, and mixtures thereof. In some embodiments, the oligonucleotide primers are single-stranded deoxyribonucleic acid (DNA) molecules. In some embodiments, primers utilized in the methods described herein to detect the presence or absence of SARS-CoV-2 are each at least 10 nucleotides in length. In some embodiments, the primers are at least about 15, 20, 25, 30, 35, 40, 45, or 50 nucleotides in length. Preferably, the guanine/cytosine (GC) ratio of the primer sequence is above 30%, above 35%, above 40%, above 45%, above 50%, above 55%, or above 60% so as to prevent hair-pin formation of the primer. The primers utilized in the methods described herein may be prepared using any suitable method, such as conventional phosphotriester and phosphodiester methods or automated embodiments thereof so long as the primers are capable of hybridizing to their target nucleotide sequences of interest. The exact length of primer will depend on many factors, including temperature, buffer, and nucleotide composition within a reaction mixture.

Primers of the present disclosure comprise a nucleotide sequence that is complementary or substantially complementary to a “target nucleotide sequence”. In some embodiments, the target nucleotide sequence comprises a nucleotide sequence portion of SARS-CoV-2 genomic RNA, e.g., a nucleotide sequence of the TM2 gene or N gene of SARS-CoV-2. In some embodiments, the target nucleotide sequence comprises a complementary sequence of the SARS-CoV-genomic RNA, e.g., a complementary DNA (cDNA) of the SARS-CoV-2 genomic RNA formed in a reverse transcription reaction. In some embodiments, the target nucleotide sequence comprises a sequence within a primer extension product formed from a primer of the present disclosure.

The terms “complementary” and “substantially complementary” refer to base pairing between nucleotides such as, for instance, between an oligonucleotide primer and its target nucleotide sequence. Complementary nucleotides are, generally, adenine and thymine, adenosine and uracil, and guanine and cytosine. Within the context of the methods disclosed herein, the oligonucleotide primers do not require complete complementarity in order to hybridize to their target nucleotide sequence. The primer sequences disclosed herein may be modified to some extent without loss of utility as specific primers. In accordance with the methods of the present disclosure, the first and second oligonucleotide primers of the primary and secondary primer set are at least 80% complementary to their target nucleotide sequence. In some embodiments, the oligonucleotide primers disclosed herein are at least 85%, at least 90%, at least 91%, 92%, 93%, 94%, 95%, 96%, 97%, 98%, 99% or 100% complementary to their target nucleotide sequence.

As is known in the art, hybridization of complementary and partially complementary nucleic acid sequences may be obtained by adjustment of the hybridization conditions to increase or decrease stringency, i.e., by adjustment of hybridization temperature or salt content of the buffer. Minor modifications of the disclosed sequences and any necessary adjustments of hybridization conditions to maintain specificity require only routine experimentation and are within the ordinary skill in the art. In some embodiments, a primer will comprise a nucleotide sequence that hybridizes to at least about 8, at least about 10, at least about 15, or about 20 to about 40 consecutive nucleotides of a target nucleic acid (i.e., the primer sequence will hybridize to a contiguous sequence within a target nucleic acid). Nucleic acid molecules that are complementary to each other can hybridize to each other under stringent conditions that are low, moderate, and/or high.

The oligonucleotide primers as disclosed herein are not naturally occurring genomic sequences, and thus, are not products of nature. The SARS-CoV-2 genome consists of a positive-sense, single strand RNA. From the full-length genomic RNA, ORF1a and ORF1b polyproteins are directly translated (i.e., no intermediate complement of the genomic RNA is produced), while translation of some or all of the structural proteins involves the production of subgenomic RNAs via discontinuous transcription events. Since the viral genome and any subgenomic RNA fragments are comprised of ribose nucleotides (i.e., ribose sugars appended to one of cytosine, guanine, adenine, and uracil nucleobases), the oligonucleotide primers described herein, which are comprised of deoxyribose nucleotides (i.e., deoxyribose sugars appended to one of cytosine, guanine, adenine, and thymine nucleobases) are structurally unique molecules that do not exist in nature.

Oligonucleotide primer pairs as described herein are designed to delineate and amplify particular regions of the SARS-CoV-2 genome using an amplification reaction such as PCR or Real Time-PCR. These exemplary amplification reactions comprise either two or three step cycles. Two step cycles have a high temperature denaturation step followed by a hybridization/elongation step. Three step cycles comprise a denaturation step, a hybridization step, and a separate elongation step. During the hybridization step, the first and/or second oligonucleotide primers of one or more primer sets as described herein hybridize to their respective target nucleotide sequence, and during the elongation step, the primers are extended to form primer extension products. The primer extension product of one primer is designed to serve as target nucleotide sequence for the other primer of the primer set in the amplification reaction. Thus, repetition of the reaction cycles results in exponential amplification of the target region, i.e., a region of the TM2 gene and/or region of the N gene of SARS-CoV-2, encompassed by primers. This target region, defined on its 5′end by the first or second primer nucleotide sequence and defined on its 3′ end by the complement of the second or first primer nucleotide sequence, respectively, is referred to herein as the amplification product or amplicon. In some embodiments, the amplification products generated in accordance with the methods described here are nucleic acid molecules that are at least 20 nucleotides in length. In some embodiments, the amplification products are 25, 30, 35, 40, 45, 50, 55, 60, 65, 70, 75, 80, 85, 90, 95, 100, or greater than 100 nucleotides in length.

Various nucleic acid amplification reactions are well known in the art and suitable for use in the methods of the present disclosure. These nucleic acid amplification reactions include, without limitation, PCR (U.S. Pat. No. 5,219,727, which is hereby incorporated by reference in its entirety) as described above and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773, which is hereby incorporated by reference in its entirety), self-sustained sequence replication and its variants (Guatelli et al. “Isothermal, In vitro Amplification of Nucleic Acids by a Multienzyme Reaction Modeled after Retroviral Replication,” Proc Natl Acad Sci USA 87(5): 1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc Natl Acad Sci USA 86(4): 1173-7 (1989), which is hereby incorporated by reference in its entirety), Qb Replicase and its variants (Miele et al. “Autocatalytic Replication of a Recombinant RNA.” J Mol Biol 171(3): 281-95 (1983), which is hereby incorporated by reference in its entirety), cold-PCR (Li et al. “Replacing PCR with COLD-PCR Enriches Variant DNA Sequences and Redefines the Sensitivity of Genetic Testing.” Nat Med 14(5): 579-84 (2008), which is hereby incorporated by reference in its entirety). Depending on the amplification technique that is employed, the amplified molecules are detected during amplification (e.g., real-time PCR) or subsequent to amplification and may involve detection of labeled amplification product, detection of component comprising amplified nucleic acid, or a byproduct of the amplification process, such as a physical, chemical, luminescence, or electrical aspect, which correlates with amplification (e.g. fluorescence, pH change, heat change, etc.). Suitable nucleic acid detection assays are described in more detail below.

In some embodiments, the nucleic acid amplification reaction employed in the method of the present disclosure is a real-time PCR. Real-time PCR, which is also referred to quantitative real time polymerase chain reaction or kinetic polymerase chain reaction, is used to amplify and simultaneously quantify one or more nucleic acid molecules present in a sample. It enables both detection and quantification (as absolute number of copies or relative amount when normalized to nucleic acid input or additional normalizing genes) of a specific sequence in a sample. Real-time PCR may be combined with reverse transcription polymerase chain reaction to quantify RNAs (real-time RT-PCR). Relative concentrations of a particular nucleic acid present during the exponential phase of real-time PCR are determined by plotting fluorescence (generated with the production of an amplification product) against cycle number on a logarithmic scale. Amounts of one or more nucleic acid molecules present in the sample are determined by comparing the results to a standard curve produced by real-time PCR of serial dilutions of a known amount of nucleic acid.

In some embodiments, the amplification reaction is carried out in a “multiplex” manner to detect the presence or absence of SARS-CoV-2 in a sample. The term “multiplex” refers to multiple assays being carried out simultaneously (i.e., in one reaction tube), in which detection and analysis steps are generally performed in parallel. Within the context of the present disclosure, a multiplex assay involves the use of the primary oligonucleotide primer set as described herein in combination with one or more additional oligonucleotide primer sets, e.g., the secondary oligonucleotide primer set and a control oligonucleotide primer set as described herein to identify two or more regions of the SARS-CoV-2 RNA in a sample simultaneously.

In accordance with the methods disclosed herein, the first oligonucleotide primer of the primary primer set comprises a nucleotide sequence that is complementary to a portion of the TM2 gene of ORF1a of SARS-CoV-2. As described above, the TM2 gene has a nucleotide sequence of SEQ ID NO: 12 (corresponding to nucleotides 9663-9743 of the ORF1ab region provided above as SEQ ID NO: 10). In some embodiments, an exemplary first oligonucleotide primer of the primary primer set comprises a nucleotide sequence having at least 90% sequence identity to the nucleotide sequence of GGATACAACTAGCTACAGAGAA (SEQ ID NO: 1). As used herein, the term “sequence identity” defines the amount of continuous nucleotide residues which match exactly between two different sequences, wherein the measurement is relational to the shorter of the two sequences. In some embodiments, the first oligonucleotide primer of the primary primer set comprises a nucleotide sequence having at least 95%, at least 96%, at least 97%, and least 98%, and least 99% sequence identity to the nucleotide sequence of SEQ ID NO: 1. In some embodiments, the first oligonucleotide primer of the primary primer set comprises a nucleotide sequence of SEQ ID NO: 1.

In some embodiments, an exemplary second oligonucleotide primer of the primary primer set comprises a nucleotide sequence that is complementary to the primer extension product formed from the first oligonucleotide primer of the primary primer set as described herein. In some embodiments, the second oligonucleotide primer of the primary primer set comprises a nucleotide sequence having at least 90% sequence identity to the nucleotide sequence of CATCAGAACCTGAGTTACTGAA (SEQ ID NO: 2). In some embodiments, the second oligonucleotide primer of the primary primer set comprises a nucleotide sequence having at least 95%, at least 96%, at least 97%, and least 98%, and least 99% sequence identity to the nucleotide sequence of SEQ ID NO: 2. In some embodiments, the second oligonucleotide primer of the primary primer set comprises a nucleotide sequence of SEQ ID NO: 2.

In accordance with the methods disclosed herein, the first oligonucleotide primer of the secondary primer set comprises a nucleotide sequence that is complementary to a portion of the N gene of SARS-CoV-2 genomic RNA, i.e., complementary to a portion of the nucleotide sequence of SEQ ID NO: 11. In some embodiments, an exemplary first oligonucleotide primer of the secondary primer set comprises a nucleotide sequence having at least 90% sequence identity to the nucleotide sequence of AACGTGGTTGACCTACAC (SEQ ID NO: 4). In some embodiments, the first oligonucleotide primer of the secondary primer set comprises a nucleotide sequence having at least 95%, at least 96%, at least 97%, and least 98%, and least 99% sequence identity to the nucleotide sequence of SEQ ID NO: 4. In some embodiments, the first oligonucleotide primer of the secondary primer set comprises a nucleotide sequence of SEQ ID NO: 4.

In some embodiments, an exemplary second oligonucleotide primer of the secondary primer set comprises a nucleotide sequence that is complementary to the primer extension product formed from the first oligonucleotide primer of the secondary primer set as described herein. In some embodiments, the second oligonucleotide primer of the secondary primer set comprises a nucleotide sequence having at least 90% sequence identity to the nucleotide sequence of GCTTATTCAGCAAAATGACTTGA (SEQ ID NO: 5). In some embodiments, the second oligonucleotide primer of the secondary primer set comprises a nucleotide sequence having at least 95%, at least 96%, at least 97%, and least 98%, and least 99% sequence identity to the nucleotide sequence of SEQ ID NO: 5. In some embodiments, the second oligonucleotide primer of the secondary primer set comprises a nucleotide sequence of SEQ ID NO: 5.

In some embodiments, at least one oligonucleotide primer of the primary primer set and/or the secondary primer set comprises a detectable label. The detectable label can be covalently or non-covalently coupled to the 5′ end of the primer. Suitable detectable labels are disclosed herein. In accordance with this embodiment, the detectable label is incorporated into the amplification products formed from the first and second primers of the primer set, and the presence or absence of SARS-CoV-2 is detected by detecting labeled TM2 and/or N gene amplification products.

In some embodiments, the primary oligonucleotide primer set and/or the secondary oligonucleotide primer set as described above each further comprise an oligonucleotide probe. The term “probe” as used herein refers to an oligonucleotide that produce a detectable response upon interaction with a target nucleotide sequence. In some embodiments, the oligonucleotide probe of the primary oligonucleotide primer set as disclosed herein includes at least one reporter moiety, and a nucleotide sequence complementary to a TM2 amplification product formed from the first and second primers of the primary oligonucleotide primer set. In some embodiments, the oligonucleotide probe of the secondary oligonucleotide primer set comprises at least one reporter moiety, and a nucleotide sequence complementary to an N gene amplification product formed from the first and second primers of the secondary primer set.

In some embodiments, the oligonucleotide probes comprise a pair of moieties that form an energy transfer pair detectable upon some change of state of the probe in response to its interaction with a binding partner. In some embodiments, the oligonucleotide probes described herein comprise more than two moieties such as a fluorophore and one or more quencher moieties. In accordance with the methods of the present disclosure, the probes hybridize to complementary regions of their respective amplification products, and the presence of SARS-CoV-2 in a sample is determined by detecting the one or more reporter moieties or interaction between the reporter moieties of the oligonucleotide probes during or after the amplification reaction.

In some embodiments, the oligonucleotide probe of the primary primer set comprises a nucleotide sequence having at least 90%, at least 95%, at least 96%, at least 97%, at least 98%, at least 99%, or 100% sequence identity to a nucleotide sequence of CTGCTTGTTGTCATCTCGCAAAG (SEQ ID NO: 3). In some embodiments, the oligonucleotide probe of the secondary primer set comprises a nucleotide sequence having at least 90%, at least 95%, at least 96%, at least 97%, at least 98%, at least 99%, or 100% sequence identity to a nucleotide sequence of CCATCAAATTGGATGACAAAGATCCAAATT (SEQ ID NO: 6).

As used herein, “detectable label” or “reporter moiety” encompasses any molecule that provides a detectable signal, and that can be coupled to an oligonucleotide primer or probe as described herein. Numerous detectable labels that may be used to label nucleic acids are known in the art. Direct reporter molecules include fluorophores, chromophores, and radiophores. Non-limiting examples of fluorophores include, a red fluorescent squarine dye such as e.g., 2,4-Bis[1,3,3-trimethyl-2-indolinylidenemethyl]cyclobutenediylium-1,3-dioxolate, an infrared dye, e.g., 2,4Bis[3,3-dimethyl-2-(1H-benz[e]indolinylidenemethyl)]cyclobutenediylium-1,3-dioxolate, or an orange fluorescent squarine dye such as, e.g., 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®-R6G, BODIPY®-TMR, BODIPY®-TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™ TAMRA™, TET™, Tetramethylrhodamine, or Texas Red®. Suitable detectable labels also include indirect reporter molecules, such as biotin, which must be bound to another molecule such as streptavidin-phycoerythrin for detection. In a multiplex reaction, the reporter moiety or detectable label coupled to the primers or probes may be the same for each target nucleic acid molecule in the multiplex reaction being detected if the identities of the amplification products can be determined based on another feature, e.g., size or specific location or identity on a solid support to which they hybridize. Alternatively, the reporter moiety or detectable label coupled to the primers and probes of a multiplex reaction may be different for each different target nucleic acid molecule being detected.

In some embodiments, fluorophore/quencher-based detection systems are utilized in the methods and compositions disclosed herein. In accordance with this embodiment, the oligonucleotide probe of the primary and/or secondary oligonucleotide primer set comprises both a reporter moiety and one or more quencher moieties. The reporter and quencher moieties are in proximity to each other such that the quencher quenches the signal produced by the reporter moiety. In some embodiments, a conformational change in the nucleic acid molecule separates the reporter moiety and quencher to allow the reporter moiety to emit a detectable signal. In some embodiments, cleavage of the reporter moiety or the quencher from the nucleic acid molecule (e.g., by polymerase extension of a primer sequence) separates the reporter from the quencher to allow the reporter moiety to emit a detectable signal. Reporter moiety/quencher-based detection systems reduce background and therefore improve the sensitivity of multiplex reactions such as those disclosed herein.

In particular embodiments, molecules useful as quenchers include, but are not limited to tetramethylrhodamine (TAMRA), DABCYL (DABSYL, DABMI or methyl red) anthroquinone, nitrothiazole, nitroimidazole, malachite green, Black Hole Quenchers®, e.g., BHQ1 (Biosearch Technologies), Iowa Black® or ZEN quenchers (from Integrated DNA Technologies, Inc.) and TIDE Quenchers (e.g. TID Quencher 2 (TQ2) and TIDE Quencher 3 (TQ3)) (from AAT Bioquest). In one embodiment, the probes used in the methods described herein comprise two quencher molecules, an internal quencher and a 3′ quencher. In accordance with this embodiment, an exemplary probe of the primary primer set comprises a nucleotide sequence with a fluorescent reporter moiety on the 5′ end, an internal quencher and a 3′ quencher, e.g., FAM-CTGCTTGTT-ZEN-GTCATCTCGCAAAG-IBFQ (SEQ ID NO: 3). Similarly, an exemplary probe of the secondary primer set comprises a nucleotide sequence with a fluorescent reporter moiety on the 5′ end, an internal quencher and a 3′ quencher, e.g., FAM-CCATCAAAT-ZEN-TGGATGACAAAGATCCAAATT-IBFQ (SEQ ID NO: 6). In accordance with the above embodiments, the reporter moieties of the oligonucleotide probes of the primary and secondary oligonucleotide primer sets are the same reporter moieties. In some embodiments, the reporter moieties of the oligonucleotide probes of the first and second oligonucleotide primer sets are different reporter moieties.

The reporter and quencher moieties as described herein can be attached to a nucleic acid molecule via a covalent bond or a noncovalent interaction. In some embodiments, the report and/or quencher moiety is attached using a linking moiety. Linking moieties and methodologies for attaching reporter or quencher molecules to the oligonucleotide primers or probes as disclosed herein are well known in the art and include, without limitation, a 3′ thiol group (see e.g., Zuckerman et al, Nucleic Acids Research 15: 5305-5321 (1987), which is hereby incorporated by reference in its entirety); a 3′ sulfhydryl moiety (see e.g., Sharma et al, Nucleic Acids Research 19: 3019 (1991)); a 5′ phosphoamino group via Aminolink™ II available from Applied Biosystems, Foster City, Calif. (see e.g., Giusti et al, PCR Methods and Applications 2: 223-227 (1993), which is hereby incorporated by reference in its entirety); 3′ aminoalkylphosphoryl group (see e.g., U.S. Pat. No. 4,739,044, which is hereby incorporated by reference in its entirety); phosphoramidate linkages, a 5′ mercapto group, and a 3′ amino group (see Agrawal et al., Tetrahedron Letters, 31:1543-1546 (1990); Sproat et al, Nucleic Acids Research 15: 4837 (1987); and Nelson et al, Nucleic Acids Research 17: 7187-7194 (1989) which are hereby incorporated by reference in their entirety).

Suitable oligonucleotide primers and probe detection systems known in the art and suitable for use in the methods disclosed herein include, without limitation, fluorescent intercalation dyes, FRET-based detection methods (U.S. Pat. No. 5,945,283; PCT Publication WO 97/22719; both of which are incorporated by reference in their entireties), Scorpion probe detection systems (Thelwell et al., Nucleic Acids Research 28:3752-3761, 2000, which is hereby incorporated by reference in its entirety), Molecular Beacons (Tyagi et al., Nat. Biotechnol. 14 (3): 303-8 (1996), which is hereby incorporated by reference in its entirety), and TaqMan detection systems (Holland et al., Proc. Nat'l Acad. Sci. USA 88(16): 7276-7280 (1991), which is hereby incorporated by reference in its entirety).

Nucleic acid amplification products produced in accordance with the methods described herein can further be analyzed by any number of techniques to determine the presence of, amount of, or identity of the molecule. Non-limiting examples of these techniques include sequencing, mass determination, and base composition determination. The analysis may identify the sequence of all or a part of the amplified nucleic acid or one or more of its properties or characteristics to reveal the desired information.

In some embodiments, the methods of the present application further involve the incorporation and detection of one or more internal controls. In one embodiment, the internal control is a positive control. A suitable positive control, includes, any non-SARS-CoV RNA or cDNA sequence. For example, a non-SARS-CoV sequence can be an intrinsic component of the sample to be assayed. Alternatively, a non-SARS-CoV sequence(s) is spiked into the sample to be assayed. In one embodiment, the spiked non-SARS-CoV control template is the genomic sequence or a portion thereof of another, non-related virus. In one embodiment, the positive control is the genomic sequence or a portion thereof originating from equine arteritis virus. The positive control is amplified and detected using a control primer set. The control primer set has a first oligonucleotide primer comprising a nucleotide sequence complementary to a first portion of the control nucleic acid template and a second oligonucleotide primer comprising a nucleotide sequence complementary to an extension product formed from the first oligonucleotide primer of the control primer set. The sample containing the control template and control reagents along with the primary and secondary primer sets is subjected to one set of amplification reaction conditions, in the same reaction mixture, for the simultaneous detection of the target regions of interest, i.e., TM2 of ORF1a, N gene, and control template regions.

Another aspect of the present application is directed to an isolated oligonucleotide suitable for detecting severe acute respiratory syndrome coronavirus 2 (SARS-CoV-2), wherein the isolated oligonucleotide comprises a nucleotide sequence having at least 90%, at least 95%, at least 96%, at least 97%, at least 98%, at least 99%, or 100% sequence identity to a nucleotide sequence selected from SEQ ID NO: 1, SEQ ID NO: 2, SEQ ID NO: 3, SEQ ID NO: 4, SEQ ID NO: 5, and SEQ ID NO: 6.

As described in more detail supra, oligonucleotides of the disclosure encompass recombinant oligonucleotides and chemically synthesized oligonucleotides. These oligonucleotides can be in the form of ribonucleotides, deoxynucleotides, modified ribonucleotides, modified deoxyribonucleotides, modified phosphate-sugar-backbone oligonucleotides, nucleotide analogs, and mixtures thereof. In some embodiments, the oligonucleotides are single-stranded DNA molecules. In some embodiments, the oligonucleotides are least 10, 15, 20, 25, 30, 35, 40, 45, or 50 nucleotides in length. Preferably, the guanine/cytosine (GC) ratio of the oligonucleotides is above 30%, above 35%, above 40%, above 45%, above 50%, above 55%, or above 60% so as to prevent hair-pin structures formation. These oligonucleotides can be prepared using suitable methods, such as chemical synthesis, recombinant methods, or both.

Another aspect of the present application is directed to an oligonucleotide primer set for detecting SARS-CoV-2 transmembrane domain 2 gene. This oligonucleotide primer set comprises a first oligonucleotide primer comprising a nucleotide sequence that is complementary to a region of the TM2 gene of SARS-CoV-2, and a second oligonucleotide primer comprising a nucleotide sequence that is complementary to an extension product formed from the first oligonucleotide primer. In some embodiments, the first oligonucleotide primer comprises a nucleotide sequence having at least 90%, at least 95%, at least 96%, at least 97%, at least 98%, at least 99%, or 100% sequence identity to a nucleotide sequence selected from SEQ ID NO: 1. In some embodiments, the second oligonucleotide primer of the primer set comprises a nucleotide sequence having at least 90%, at least 95%, at least 96%, at least 97%, at least 98%, at least 99%, or 100% sequence identity to a nucleotide sequence selected from SEQ ID NO: 2. In some embodiments, the oligonucleotide primer set further comprises an oligonucleotide probe. The oligonucleotide probe comprises a nucleotide sequence that is complementary to a primer extension product of the first or second oligonucleotide primers of the primer set. In some embodiments, the oligonucleotide probe comprises a nucleotide sequence having at least 90%, at least 95%, at least 96%, at least 97%, at least 98%, at least 99%, or 100% sequence identity to a nucleotide sequence selected from SEQ ID NO: 3. As described supra, the oligonucleotide probe may comprise a reporter moiety and at least one quencher molecule. Reporter molecules and quenchers are described supra. In one embodiment, the oligonucleotide probe of this primer set comprises a 5′ fluorescent reporter moiety, an internal quencher molecule, and 3′ quencher molecule.

Another aspect of the present application is directed to an oligonucleotide primer set for detecting SARS-CoV-2 N gene. This oligonucleotide primer set comprises a first oligonucleotide primer comprising a nucleotide sequence that is complementary to a region of the N gene of SARS-CoV-2, and a second oligonucleotide primer comprising a nucleotide sequence that is complementary to an extension product formed from the first oligonucleotide primer. In some embodiments, the first oligonucleotide primer comprises a nucleotide sequence having at least 90%, at least 95%, at least 96%, at least 97%, at least 98%, at least 99%, or 100% sequence identity to a nucleotide sequence selected from SEQ ID NO: 4. In some embodiments, the second oligonucleotide primer of the primer set comprises a nucleotide sequence having at least 90%, at least 95%, at least 96%, at least 97%, at least 98%, at least 99%, or 100% sequence identity to a nucleotide sequence selected from SEQ ID NO: 5. In some embodiments, the oligonucleotide primer set further comprises an oligonucleotide probe. The oligonucleotide probe comprises a nucleotides sequence that is complementary to a primer extension product of the first or second oligonucleotide primers of the primer set. In some embodiments, the oligonucleotide probe comprises a nucleotide sequence having at least 90%, at least 95%, at least 96%, at least 97%, at least 98%, at least 99%, or 100% sequence identity to a nucleotide sequence selected from SEQ ID NO: 6. As described supra, the oligonucleotide probe may comprise a reporter moiety and at least one quencher molecule. Reporter molecules and quenchers are described supra. In one embodiment, the oligonucleotide probe of this primer set comprises a 5′ fluorescent reporter moiety, an internal quencher molecule, and 3′ quencher molecule.

The invention also encompasses kits for detecting the presence of SARS-CoV-2 in a test sample. Suitable amplification reaction reagents that can be included in a kit may include, for example, one or more of buffers, an enzyme having reverse transcriptase activity, an enzyme having polymerase activity, enzyme cofactors such as magnesium or manganese, salts, nicotinamide adenine dinucleotide (NAD), and deoxynucleoside triphosphates (dNTPs) such as, for example, deoxyadenosine triphosphate, deoxyguanosine triphosphate, deoxycytidine triphosphate and deoxythymidine triphosphate, biotinylated dNTPs, suitable for carrying out the amplification reactions.

Depending on the procedure, the kit may further comprise one or more of: wash buffers and/or reagents, hybridization buffers and/or reagents, labeling buffers and/or reagents, and detection means. The buffers and/or reagents included in a kit are preferably optimized for the particular amplification/detection technique for which the kit is intended. Protocols for using these buffers and reagents for performing different steps of the procedure may also be included in the kit.

In some embodiments, the kit comprises a positive control. In some embodiments, a kit comprises a negative control. In some embodiments, a negative control comprises any sequence not subject to amplification by primers useful for the amplification and detection of the TM2 gene of ORF1a or the N gene. Furthermore, the kits may be provided with an internal control as a check on the amplification procedure and to prevent occurrence of false negative test results due to failures in the amplification procedure. An optimal internal control sequence is selected in such a way that it will not compete with amplification and detection of the SARS-CoV-2 target nucleic acid molecules in the amplification reaction. In some embodiments, the internal control may be a sequence originating from a different virus, e.g., the nucleotide sequence encoding equine arteritis virus or one or more genes of the equine arteritis virus.

Kits may also contain reagents for the isolation of nucleic acids from a sample prior to amplification, for example reagents suitable for isolating genomic RNA from the sample.

The reagents may be supplied in a solid (e.g., lyophilized) or liquid form. The kits of the present disclosure optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents, primer sets, and/or primer/probe sets according to the present disclosure. Instructions for using the kit according to one or more methods of the present disclosure may comprise instructions for processing the biological sample, extracting nucleic acid molecules, and/or performing the test; instructions for interpreting the results as well as a notice in the form prescribed by a governmental agency (e.g., FDA) regulating the manufacture, use or sale of test reagents and results.

In one embodiment, the kit comprises an oligonucleotide suitable for detecting severe acute respiratory syndrome coronavirus 2 (SARS-CoV-2) as described supra. In some embodiments, the kit comprises the oligonucleotide primer set for detecting SARS-CoV-2 TM2 gene as described supra. In some embodiments, the kit comprises the oligonucleotide primer set for detecting SARS-CoV-2 N gene as described supra. In some embodiments, the kit comprises the oligonucleotide primer set for detecting SARS-CoV-2 TM2 gene and the oligonucleotide primer set for detecting the SARS-CoV-2 N gene as described supra.

In one embodiment, the kit comprises one or more reagents for carrying out a real-time reverse transcription polymerase chain reaction. Exemplary reagents include, without limitation, the primers and probes described herein, an enzyme mix comprising a reverse transcriptase and a DNA polymerase, as well as suitable buffers for the reaction.

### EXAMPLES

### Example 1: Targeted Regions and Assay Design

The following primers and probes are provided which hybridize to target regions present in SARS-Cov-2 genome to form detectable probe/target hybrids indicating the presence of SARS-CoV-2 in a test sample.

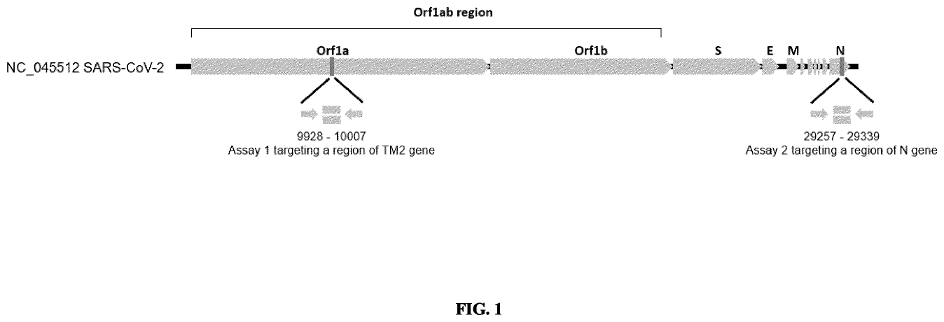

The two SARS-CoV-2 genomic regions targeted by the two different SARS-CoV-2 primer and probe sets are located in ORF1ab region (region coding for the Transmembrane domain 2 (TM2) gene of ORF1a) and in the N gene (coding for the Nucleocapsid phosphoprotein) (FIG. 1). The expected size of the TM2 amplicon is 80 base pairs and the expected size of the N gene amplicon is 83 base pairs.

Both specific probes of SARS-CoV-2 feature a 5′fluorescent reporter FAM dye, an internal ZEN® quencher located between the 9th and 10th base from the reporter FAM dye on the 5′ end of the probe sequence, and a 3′ Iowa Black® dark quencher (IBFQ). The advantage of having an internal quencher is to reduce the distance between the FAM dye and the quencher, and thus in combination with the terminal 3′ quencher, provides a higher degree of quenching and lowers initial background. Having both specific SARS-CoV-2 probes on the same FAM detecting channel is thought to prevent false negative results due to probe binding failure and to guaranty inclusivity of the assay. It also improves the reaction of fluorescence as the second identically labelled probe has an additive effect on the overall reaction fluorescence compared to the single probe assay. This higher fluorescence facilitates the evaluation and interpretation of weak positive specimens (Nagy et al., “Evaluation of TaqMan qPCR System Integrating Two Identically Labelled Hydrolysis Probes in Single Assay,” Scientific Reports 7:41392 (2017); Yip et al., “Use of Dual TaqMan Probes to Increase the Sensitivity of 1-Step Quantitative Reverse Transcription-PCR: Application to the Detection of SARS Coronavirus,” Clinical Chemistry 51(10):1885-1888 (2005), which are hereby incorporated by reference in their entirety).

The mix further includes a primer and probe set to detect a sequence located in the genome of equine arteritis virus (EAV) that serves as internal control (Region targeted of EAV assay (NC_002532): 1843-1976). Specific probe of EAV feature 5′fluorescent reporter CY5 dye and a 3′ Iowa Black® dark quencher (IBRQ).

The table below includes the nucleic acid sequences for all primers and probes used in the assay described herein.

TABLE 1

| | | | Final | SEQ |
|---|---|---|---|---|
| | primer/ | | concentration | ID |
| description | probe | sequences | (nM) | NO: |
| SARS-CoV- | forward | AACGTGGTTGA | 700 | 4 |
| 2_N_F | primer | CCTACAC | | |
| SARS-CoV- | probe | FAM-CCATCA | 200 | 6 |
| 2_N_P | | AAT-ZEN- | | |
| | | TGGATGACAA | | |
| | | AGATCCAAAT | | |
| | | T-IBFQ | | |
| SARS-CoV- | reverse | GCTTATTCAG | 700 | 5 |
| 2_N_R | primer | CAAAATGACT | | |
| | | TGA | | |
| SARS-CoV- | forward | GGATACAACT | 500 | 1 |
| 2 ORF1ab_F | primer | AGCTACAGAG | | |
| | | AA | | |
| SARS-CoV- | probe | FAM-CTGCTT | 100 | 3 |
| | | GTT-ZEN-GT | | |
| 2 ORF1ab_P | | CATCTCGCAA | | |
| | | AG-IBFQ | | |
| SARS-CoV- | reverse | CATCAGAACC | 500 | 2 |
| 2 ORF1ab_R | primer | TGAGTTACTG | | |
| | | AA | | |
| EAV_F | forward | CATCTCTTGC | 500 | 7 |
| | primer | TTTGCTCCTT | | |
| | | AG | | |
| EAV_P | probe | CGCTGTCAGA | 100 | 9 |
| | | ACAACATTAT | | |
| | | TGCCCAC | | |
| EAV_R | reverse | Cy5-AGCCGC | 500 | 8 |
| | primer | ACCTTCACAT | | |
| | | TG-IBRQ | | |

These primers and probes preferentially hybridize to the target nucleic acid derived from SARS-CoV-2 and equine arteritis virus, respectively, under strict hybridization assay conditions.

### Example 2: Extraction Procedure

Nucleic acids are isolated and purified from human nasopharyngeal swabs using a commercially available nucleic acid extraction kit for automated extraction with a sample input volume of 200 μL and an elution volume of 55 μL. 2 μL of internal control (IC) is added to each sample and negative control (NC) during the extraction process.

### Example 3: qPCR Procedure

A total of 10 μL of the purified nucleic acid is added to a real time RT-PCR reaction mix consisting of primer and probe mix (1.5 μL) (see Table 1 for concentrations used), enzyme mix (RT enzyme and Taq polymerase) (1 μL) and buffer (12.5 μL) and reverse transcribed into cDNA which is then subsequently amplified in an Applied Biosystems® 7500 Real-Time PCR thermocycler. The buffer composition includes Tris, Potassium Chloride, Magnesium Chloride, dATP, dCTP, dGTP, dTTP, recombinant albumin, Trehalose with a pH 8.7. The cycling run profile can be found below. As the assay is a multiplex PCR detecting two genomic regions of SARS-CoV-2 and the internal control, all three targets are amplified at the same time.

The PCR program is as follows: 50° C. for 15 minutes hold, 94° C. for 1-minute hold, 40 cycles of: 94° C. for 8 seconds, and 60° C. for 1 minute.

### Example 4: Clinical Performance Assessment

The clinical performance of the SAR-CoV-2 detection assay disclosed herein was established using prospectively collected nasopharyngeal swabs (NPS) and oropharyngeal swabs (OPS). A total of 101 specimens were collected from symptomatic patients with suspicion of COVID-19. The clinical performance study was conducted in a diagnostic laboratory and was evaluated by comparing the results of the method disclosed herein (carried out per the methods of Examples 2 and 3 above) with results obtained using a commercially available SARS-CoV-2 nucleic acid amplification kit (CE-IVD nucleic acid amplification test (NAAT)). The results (summarized in the Tables below) showed an overall diagnostic sensitivity of 100% (95% Confidence Interval: 91.78-100) and an overall diagnostic specificity of 100% (95% Confidence Interval: 93.84-100) for the detection of SARS-CoV-2 in both matrices using SARS-CoV-2 detection assay disclosed herein.

| | Diagnostic | Pass/fail | Diagnostic | Pass/fail |
|---|---|---|---|---|
| | sensitivity | acceptance | specificity | acceptance |
| Pathogen | in % (95% CI) | criteria | in % (95% CI) | criteria |
| SARS-CoV-2 | 100% | PASS | 100% | |
| | (91.8-100) | | (93.8-100) | |

Diagnostic sensitivity and specificity obtained by the SARS-CoV-2 detection assay disclosed herein

| | | Diagnostic | | | Diagnostic | | |
|---|---|---|---|---|---|---|---|
| | | sensitivity | | 95% | specificity | | 95% |
| | Sample | | Total | Confidence | | Total | Confidence |
| Pathogen | type | Percentage | number | interval | Percentage | number | interval |
| SARS-CoV-2 | NPS | 100% | 12/12 | 73.5-100 | 100% | 3/3 | 29.2-100 |
| | OPS | 100% | 31/31 | 88.8-100 | 100% | 55/55 | 93.5-100 |
| | Overall | 100% | 43/43 | 91.8-100 | 100% | 58/58 | 93.8-100 |

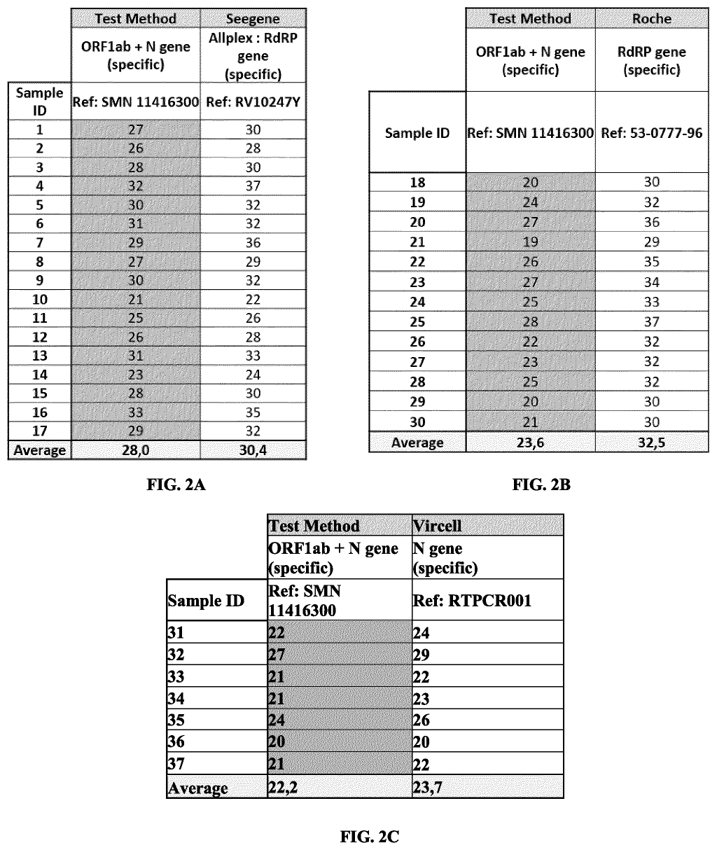

A comparative study between SARS-CoV-2 specific assays was conducted to determine sensitivity of the method disclosed herein. In particular, the SARS-CoV-2 detection assay disclosed herein (“Test Method”) was compared to SARS-CoV-2 assays from Seegene (FIG. 2A), Roche (FIG. 2B), and Vircell (FIG. 2C). Individual clinical positive samples were compared and values displayed in the tables of FIGS. 2A-2C correspond to the cycle threshold (Ct) values, i.e., the number of cycles required for the fluorescent signal to exceed background level. When comparing Ct values of the SARS-CoV-2 assay disclosed herein to commercially available SARS-CoV-2 detection assays, the detection assay disclosed herein always produced lower Ct values, indicating better sensitivity per clinical sample than the Ct values obtained with the commercially available SARS-CoV-2 detection assays.

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application as defined in the claims which follow.

1. A method for detecting presence or absence of severe acute respiratory syndrome coronavirus 2 (SARS-CoV-2) in a sample, said method comprising:
contacting the sample with a primary oligonucleotide primer set, wherein said primary oligonucleotide primer set comprises:
(i) a first oligonucleotide primer comprising a nucleotide sequence complementary to a first portion of the SARS-CoV-2 transmembrane domain 2 gene of ORF1a, and
(ii) a second oligonucleotide primer comprising a nucleotide sequence complementary to an extension product formed from the first oligonucleotide primer of the primary oligonucleotide primer set;
amplifying from the sample with the primary oligonucleotide primer set in an amplification reaction under conditions suitable for producing transmembrane domain 2 gene amplification products; and
detecting the presence or absence of SARS-CoV-2 in the sample based on said amplifying.

2. The method of claim 1, wherein at least one oligonucleotide primer of the primary primer set comprises a detectable label, and said detecting comprises detecting labeled transmembrane domain 2 gene amplification products.

3. The method of claim 1, wherein the primary oligonucleotide primer set further comprises an oligonucleotide probe, wherein said oligonucleotide probe comprises a reporter moiety and a nucleotide sequence complementary to a transmembrane domain 2 gene amplification product, wherein said probe hybridizes to its complementary nucleotide sequence of the transmembrane domain 2 gene amplification product during said amplifying, and said detecting comprises detecting the reporter moiety of the oligonucleotide probe of the primary oligonucleotide primer set during said amplifying.

4. The method of claim 1, wherein said contacting further comprises:
contacting the sample with a secondary oligonucleotide primer set, wherein said secondary oligonucleotide primer set comprises:
(i) a first oligonucleotide primer comprising a nucleotide sequence complementary to a first portion of the SARS-CoV-2 N gene, and
(ii) a second oligonucleotide primer comprising a nucleotide sequence complementary to an extension product formed from the first oligonucleotide primer of the secondary oligonucleotide primer set, wherein N gene and transmembrane domain 2 gene amplification products are produced during said amplifying.

5. The method of claim 4, wherein the secondary oligonucleotide primer set further comprises an oligonucleotide probe, wherein said oligonucleotide probe of the secondary oligonucleotide primer set comprises a reporter moiety and a nucleotide sequence complementary to an N gene amplification product, wherein said probe hybridizes to its complementary nucleotide sequence of the N gene amplification product during said amplifying, and said detecting comprises detecting one or both reporter moieties of the oligonucleotide probes of the primary and secondary oligonucleotide primer sets during said amplifying.

6. The method of claim 5, wherein the reporter moieties of the oligonucleotide probes of the first and second oligonucleotide primer sets are the same reporter moieties.

7. The method of claim 5 wherein the reporter moieties of the oligonucleotide probes of the first and second oligonucleotide primer sets are different reporter moieties.

8. The method of claim 3, wherein the reporter moieties of the oligonucleotide probes of the first and second oligonucleotide primer sets comprise fluorescent molecules.

9. The method of claim 1, wherein said amplifying further comprises:
subjecting the sample to a reverse transcription reaction prior to said amplification reaction.

10. The method of claim 1, wherein the first oligonucleotide primer of the primary primer set comprises a nucleotide sequence having at least 90% sequence identity to the nucleotide sequence of SEQ ID NO: 1, and the second oligonucleotide primer of the primary primer set comprises a nucleotide sequence having at least 90% sequence identity to the nucleotide sequence of SEQ ID NO: 2.

11. The method of claim 4, wherein the first oligonucleotide primer of the secondary primer set comprises a nucleotide sequence having at least 90% sequence identity to the nucleotide sequence of SEQ ID NO: 4, and the second oligonucleotide primer of the secondary primer set comprises a nucleotide sequence having at least 90% sequence identity to the nucleotide sequence of SEQ ID NO: 5.

12. The method of claim 3, wherein the oligonucleotide probe of the primary primer set comprises a nucleotide sequence having at least 90% sequence identity to the nucleotide sequence of SEQ ID NO: 3.

13. The method of claim 5, wherein the oligonucleotide probe of the secondary primer set comprises a nucleotide sequence having at least 90% sequence identity to the nucleotide sequence of SEQ ID NO: 6.

14. The method of claim 1, wherein the amplification reaction is a real-time reverse transcription polymerase chain reaction.

15. The method of claim 1, wherein the sample is selected from a nasopharyngeal sample, an oropharyngeal sample, and a saliva sample.

16. An oligonucleotide or an oligonucleotide primer set suitable for detecting severe acute respiratory syndrome coronavirus 2 (SARS-CoV-2), wherein said oligonucleotide or said oligonucleotide primer set comprises a nucleotide sequence having at least 90% sequence identity to a nucleotide sequence selected from the group consisting of SEQ ID NO: 1, SEQ ID NO: 2, SEQ ID NO: 3, SEQ ID NO: 4, SEQ ID NO: 5, and SEQ ID NO: 6.

17.-19. (canceled)

20. The oligonucleotide primer set of claim 16 for detecting SARS-CoV-2 N gene, said oligonucleotide primer set comprising:
a first oligonucleotide primer comprising a nucleotide sequence having at least 90% sequence identity to the nucleotide sequence of SEQ ID NO: 4, and
a second oligonucleotide primer comprising a nucleotide sequence having at least 90% sequence identity to the nucleotide sequence of SEQ ID NO: 5.

21. The oligonucleotide primer set of claim 20 further comprising:
an oligonucleotide probe comprising a nucleotide sequence having at least 90% sequence identity to the nucleotide sequence of SEQ ID NO: 6.

22. The oligonucleotide primer set of claim 20, wherein said oligonucleotide probe comprises a reporter moiety and at least one quencher molecule.

23. A kit for detecting severe acute respiratory syndrome coronavirus 2 (SARS-CoV-2), said kit comprising: an isolated oligonucleotide of claim 16, an oligonucleotide primer set of claim 16, or any combination thereof.

24.-25. (canceled)


SEQUENCE LISTING (12 sequences)

SEQ ID NO: 1 - DNA, 22 bp, Artificial
  primer
ggatacaactagctacagagaa

SEQ ID NO: 2 - DNA, 22 bp, Artificial
  primer
catcagaacctgagttactgaa

SEQ ID NO: 3 - DNA, 23 bp, Artificial
  primer
ctgcttgttgtcatctcgcaaag

SEQ ID NO: 4 - DNA, 18 bp, Artificial
  primer
aacgtggttgacctacac

SEQ ID NO: 5 - DNA, 23 bp, Artificial
  primer
gcttattcagcaaaatgacttga

SEQ ID NO: 6 - DNA, 30 bp, Artificial
  primer
ccatcaaattggatgacaaagatccaaatt

SEQ ID NO: 7 - DNA, 22 bp, Artificial
  primer
catctcttgctttgctccttag

SEQ ID NO: 8 - DNA, 18 bp, Artificial
  primer
agccgcaccttcacattg

SEQ ID NO: 9 - DNA, 27 bp, Artificial
  primer
cgctgtcagaacaacattattgcccac

SEQ ID NO: 10 - RNA, 21291 nt, Betacoronavirus severe acute respiratory syndrome- related coronavirus
auggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauuauuuuggcaucuuuuucugcuuccacaagugcuuuuguggaaacugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguugacuucgcaguggcuaacuaacaucuuuggcacuguuuaugaaaaacucaaacccguccuugauuggcuugaagagaaguuuaaggaagguguagaguuucuuagagacgguugggaaauuguuaaauuuaucucaaccugugcuugugaaauugucgguggacaaauugucaccugugcaaaggaaauuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacuggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuauguugcucgaaaucaaagacacagaaaaguacugugcccuugcaccuaauaugaugguaacaaacaauaccuucacacucaaaggcggugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguauggcuacauacuacuuauuugaugagucuggugaguuuaaauuggcuucacauauguauuguucuuucuacccuccagaugaggaugaagaagaaggugauugugaagaagaagaguuugagccaucaacucaauaugaguaugguacugaagaugauuaccaagguaaaccuuuggaauuuggugccacuucugcugcucuucaaccugaagaagagcaagaagaagauugguuagaugaugauagucaacaaacuguuggucaacaagacggcagugaggacaaucagacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagcuaaaaagguaaaaccaacagugguuguuaaugcagccaauguuuaccuuaaacauggaggagguguugcaggagccuuaaauaaggcuacuaacaaugccaugcaaguugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugacccuauacauucuuuaagaguuuguguagauacuguucgcacaaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacucuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggaggcaaagacagugcuuaaaaaguguaaaagugccuuuuacauucuaccaucuauuaucucuaaugagaagcaagaaauucuuggaacuguuucuuggaauuugcgagaaaugcuugcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaagcaugguacauuuacuugugcuagugaguacacugguaauuaccaguguggucacuauaaacauauaacuucuaaagaaacuuuguauugcauagacggugcuuuacuuacaaaguccucagaauacaaagguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauccaaacgcaagcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagcacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaaccagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuacucaugguuuagcugcuguuaauagugucccuugggauacuauagcuaauuaugcuaagccuuuucuuaacaaaguuguuaguacaacuacuaacauaguuacacgguguuuaaaccguguuuguacuaauuauaugccuuauuucuuuacuuuauugcuacaauuguguacuuuuacuagaaguacaaauucuagaauuaaagcaucuaugccgacuacuauagcaaagaauacuguuaagagugucgguaaauuuugucuagaggcuucauuuaauuauuugaagucaccuaauuuuucuaaacugauaaauauuauaauuugguuuuuacuauuaaguguuugccuagguucuuuaaucuacucaaccgcugcuuuagguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguaauucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagguucauugccuauuaauguuauaguuuuugaugguaaaucaaaaugugaagaaucaucugcaaaaucagcgucuguuuacuacagucagcuuaugugucaaccuauacuguuacuagaucaggcauuagugucugauguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacaugacaccccgugaccuuggugcuuguauugacuguagugcgcgucauauuaaugcgcagguagcaaaaagucacaacauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuugacacaugguuuagccagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacuuugcaacaucagcuuguguuuuggcugcugaauguacaauuuuuaaagaugcuucugguaagccaguaccauauuguuaugauaccaauguacuagaagguucuguugcuuaugaaaguuuacgcccugacacacguuaugugcucauggauggcucuauuauucaauuuccuaacaccuaccuugaagguucuguuagagugguaacaacuuuugauucugaguacuguaggcacggcacuugugaaagaucagaagcugguguuuguguaucuacuagugguagauggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagcuuuuggugaauacagucauguaguugccuuuaauacuuuacuauuccuuaugucauucacuguacucuguuuaacaccaguuuacucauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagcaauggauacaacuagcuacagagaagcugcuuguugucaucucgcaaaggcucucaaugacuucaguaacucagguucugauguucuuuaccaaccaccacaaaccucuaucaccucagcuguuuugcagagugguuuuagaaaaauggcauucccaucugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauucguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaacauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuuguaugaaaaugccuuuuuaccuuuugcuauggguauuauugcuaugucugcuuuugcaaugauguuugucaaacauaagcaugcauuucucuguuuguuuuuguuaccuucucuugccacuguagcuuauuuuaauauggucuauaugccugcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuugacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuacucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaaugacauucucuuagcuaaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaaccggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaucucucugacagagucguauuugucuuaugggcacauggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaucuguauugucaaguccaugguaaugcacauguagcuaguugugaugcaaucaugacuaggugucuagcuguccacgagugcuuuguuaagcguguugacuggacuauugaauauccuauaauuggugaugaacugaagauuaaugcggcuuguagaaagguucaacacaugguuguuaaagcugcauuauuagcagacaaauucccaguucuucacgacauugguaacccuaaagcuauuaaguguguaccucaagcugauguagaauggaaguucuaugaugcacagccuuguagugacaaagcuuauaaaauagaagaauuauucuauucuuaugccacacauucugacaaauucacagaugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuacaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaa

SEQ ID NO: 11 - RNA, 1260 nt, Betacoronavirus severe acute respiratory syndrome- related coronavirus
augucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaa

SEQ ID NO: 12 - RNA, 81 nt, Betacoronavirus severe acute respiratory syndrome- related coronavirus
ggauacaacuagcuacagagaagcugcuuguugucaucucgcaaaggcucucaaugacuucaguaacucagguucugaugu